(19) United States
(12) Patent Application Publication
(10) Pub. No.: US 2024/0293939 A1
(43) Pub. Date: Sep. 5, 2024

(54) ROBOTIC SURGICAL CONTROL AND NAVIGATION

(71) Applicant: Zeta Surgical Inc., Boston, MA (US)

(72) Inventors: Raahil Mohammed SHA, Allen, TX (US); Avinash LAL, Woodhaven, NY (US); Benjamin Hoyoung LEE, Allston, MA (US); Jose Maria Amich MANERO, Cambridge, MA (US)

(21) Appl. No.: 18/597,670

(22) Filed: Mar. 6, 2024

Related U.S. Application Data
(63) Continuation of application No. PCT/US2022/042659, filed on Sep. 6, 2022.
(60) Provisional application No. 63/241,285, filed on Sep. 7, 2021.
(60) Provisional application No. 63/355,497, filed on Jun. 24, 2022.

(51) Int. Cl. B25J 9/16; A61B 34/20; A61B 34/32; A61B 90/00; B25J 13/08; G06T 7/73

(52) U.S. Cl. CPC B25J 9/1697; A61B 34/20; A61B 34/32; A61B 90/37; B25J 9/1692; B25J 13/089; G06T 7/74; A61B 2034/2065; G06T 2207/10028; G06T 2207/30196; G06T 2207/30204

(57) ABSTRACT

Systems and methods for controlling and navigating robots in a surgical environment are disclosed. The systems and methods described herein provide techniques to adjust the location of a robot, such as a surgical robot, in response to detecting movement of a patient using image-based tracking techniques. Techniques are provided that enables a robot control system to adjust a position of a surgical robot in real-time or near real-time in response to measurements from sensors coupled to the robot or a patient in a surgical environment. Techniques for initiating a collaborative control status of a surgical robot in response to detecting image alignment errors, sensor measurements, or other conditions are disclosed.

### CROSS-REFERENCE TO RELATED APPLICATIONS

The present application is a continuation of International Patent Application No. PCT/US2022/042659 filed on Sep. 6, 2022, which claims the benefit of U.S. Provisional Patent Application No. 63/241,285, filed on Sep. 7, 2021, and claims the benefit of and priority to U.S. Provisional Patent Application No. 63/355,497, filed on Jun. 24, 2022, the contents of each of which are incorporated by reference herein in their entirety and for all purposes.

### BACKGROUND

Positioning surgical tools within a patient can be challenging. Surgical robots can be provided in surgical environments to aid in carrying out procedures.

### SUMMARY

The present disclosure relates generally to the field of surgical robot navigation and control for invasive and non-invasive surgical procedures. The present solution provides techniques for tracking movement of a patient within a surgical environment, and adjusting or navigating a surgical robot to carry out predetermined procedures while compensating for the patient movement. The techniques described herein can be implemented using a variety of movement detection techniques, such as patient tracking techniques or torque sensing techniques. The present disclosure further provides techniques to initiating collaborative control of a surgical robot by switching to manual control over surgical tools during a surgical procedure in response to various conditions. The present solution can be further used for non-invasive surgical navigation, such as transcranial magnetic stimulation (TMS) and focused ultrasound (FUS) by combining the image guidance of the present solution with surgical instruments. The present solution allows for robotic control of the surgical instrument for both invasive and non-invasive cranial procedures and utilizes the real-time registration to target highlighted locations of interest.

At least one aspect of the present disclosure is directed to a method for controlling a robot using image-based tracking techniques. The method can include accessing a three-dimensional (3D) point cloud corresponding to a surgical environment and a patient, the 3D point cloud having a frame of reference. The method can include determining a position of a surgical robot within the frame of reference of the 3D point cloud. The method can include detecting a change in a position of the patient based on a corresponding change in position of one or more points in the 3D point cloud. The method can include generating, responsive to detecting the change in the position of the patient, instructions to modify the position of the surgical robot based on the change in position of the one or more points.

In some implementations, determining the position of the surgical robot within the frame of reference can include calibrating the surgical robot using a calibration technique. In some implementations, the surgical robot further comprises a display positioned over a surgical site in the surgical environment. In some implementations, the method can include presenting an image captured by a capture device mounted on the surgical robot. In some implementations, the surgical robot can include an attachment that receives a surgical tool. In some implementations, determining the position of the surgical robot can include determining a position of the surgical tool.

In some implementations, the method can include comprising navigating the surgical robot along a predetermined pathway in the frame of reference. In some implementations, navigating the surgical robot can include adjusting a position of the surgical robot according to a predetermined trajectory in the frame of reference. In some implementations, navigating the surgical robot can include periodically determining whether the change in the position of the patient satisfies a threshold. In some implementations, navigating the surgical robot can include adjusting the position of the surgical robot according to the predetermined trajectory and the change in the position of the patient responsive to determining that the change in the position of the patient satisfies the threshold.

In some implementations, determining the position of the surgical robot is based on an infrared tracking technique. In some implementations, the surgical robot comprises one or more markers. In some implementations, determining the position of the surgical robot based on the infrared tracking technique comprises detecting a respective position of each of the one or more markers. In some implementations, detecting the change in the position of the patient comprises comparing a point of the 3D point cloud with a second point of a second 3D point cloud captured after the 3D point cloud. In some implementations, detecting the change in the position of the patient comprises determining that a distance between the point and the second point exceeds a predetermined threshold.

At least one aspect of the present disclosure is directed to a system for controlling a robot using image-based tracking techniques. The system can include one or more processors coupled to a non-transitory memory. The system can access a 3D point cloud corresponding to a surgical environment and a patient. The 3D point cloud can have a frame of reference. The system can determine a position of a surgical robot within the frame of reference of the 3D point cloud. The system can detect a change in a position of the patient based on a corresponding change in position of one or more points in the 3D point cloud. The system can generate, responsive to detecting the change in the position of the patient, instructions to modify the position of the surgical robot based on the change in position of the one or more points.

In some implementations, the system can determine the position of the surgical robot within the frame of reference by performing operations comprising calibrating the surgical robot using a calibration technique. In some implementations, the surgical robot further comprises a display positioned over a surgical site in the surgical environment. In some implementations, the system can present an image captured by a capture device mounted on the surgical robot. In some implementations, the surgical robot can include an attachment that receives a surgical tool. In some implementations, the system can determine a position of the surgical tool. In some implementations, the system can navigate the surgical robot along a predetermined pathway in the frame of reference.

In some implementations, to navigate the surgical robot, the system can adjust a position of the surgical robot according to a predetermined trajectory in the frame of reference. In some implementations, to navigate the surgical robot, the system can periodically determine whether the change in the position of the patient satisfies a threshold. In some implementations, to navigate the surgical robot, the system can adjust the position of the surgical robot according to the predetermined trajectory and the change in the position of the patient responsive to determining that the change in the position of the patient satisfies the threshold.

In some implementations, the system can determine the position of the surgical robot based on an infrared tracking technique. In some implementations, the surgical robot comprises one or more markers. In some implementations, the system can detect a respective position of each of the one or more markers. In some implementations, the system can detect the change in the position of the patient by performing operations comprising comparing a point of the 3D point cloud with a second point of a second 3D point cloud captured after the 3D point cloud. In some implementations, the system can detect the change in the position of the patient by performing operations comprising determining that a distance between the point and the second point exceeds a predetermined threshold.

At least one other aspect of the present disclosure is directed to a method of controlling a robot based on torque sensing techniques. The method can include identifying a set of measurements captured by one or more torque sensors in a surgical environment including a patient. The method can include determining a position of a surgical robot within the surgical environment. The method can include detecting a position modification condition based on the set of measurements captured by the one or more torque sensors. The method can include generating, responsive to detecting the position modification condition, instructions to modify the position of the surgical robot based on the set of measurements.

In some implementations, the one or more torque sensors are coupled to the patient. In some implementations, the method can include detecting the position modification condition further comprises determining that movement of the patient satisfies a predetermined threshold. In some implementations, the one or more torque sensors are coupled to the surgical robot. In some implementations, the method can include detecting the position modification condition further comprises determining that a collision occurred with the surgical robot based on the set of measurements. In some implementations, the one or more torque sensors are coupled to the surgical robot.

In some implementations, the method can include detecting the position modification condition further comprises determining that a position of the surgical robot has deviated from a predetermined trajectory based on the set of measurements. In some implementations, the surgical robot can include a display positioned over a surgical site in the surgical environment. In some implementations, the method can include presenting a view of the patient in the surgical environment on the display. In some implementations, the surgical robot can include an attachment that receives a surgical tool. In some implementations, determining the position of the surgical robot can include determining a position of the surgical tool.

In some implementations, the one or more torque sensors comprise at least one of an accelerometer, a gyroscope, or an inertial measurement unity (IMU). In some implementations, determining the position of the surgical robot is based on an infrared tracking technique. In some implementations, the surgical robot comprises one or more markers. In some implementations, the method can include determining a respective position of each of the one or more markers. In some implementations, generating the instructions to modify the position of the surgical robot can include generating the instructions to move the surgical robot according to movement of the patient.

At least one other aspect of the present disclosure is directed to a system for controlling a robot based on torque sensing techniques. The system can include one or more processors coupled to a non-transitory memory. The system can identify a set of measurements captured by one or more torque sensors in a surgical environment including a patient. The system can determine a position of a surgical robot within the surgical environment. The system can detect a position modification condition based on the set of measurements captured by the one or more torque sensors. The system can generate, responsive to detecting the position modification condition, instructions to modify the position of the surgical robot based on the set of measurements.

In some implementations, the one or more torque sensors are coupled to the patient. In some implementations, the system can detect the position modification condition by performing operations comprising determining that movement of the patient satisfies a predetermined threshold. In some implementations, the one or more torque sensors are coupled to the surgical robot. In some implementations, the system can detect the position modification condition by performing operations comprising determine that a collision occurred with the surgical robot based on the set of measurements. In some implementations, the one or more torque sensors are coupled to the surgical robot. In some implementations, the system can detect the position modification condition by performing operations comprising determine that a position of the surgical robot has deviated from a predetermined trajectory based on the set of measurements.

In some implementations, the surgical robot further comprises a display positioned over a surgical site in the surgical environment. In some implementations, the system can present a view of the patient in the surgical environment on the display. In some implementations, the surgical robot comprises an attachment that receives a surgical tool. In some implementations, the system can determine the position of the surgical robot by performing operations comprising determining a position of the surgical tool. In some implementations, the one or more torque sensors can include at least one of an accelerometer, a gyroscope, or an inertial measurement unity (IMU).

In some implementations, the system can determine the position of the surgical robot is based on an infrared tracking technique. In some implementations, the surgical robot comprises one or more markers. In some implementations, the system can determine a respective position of each of the one or more markers. In some implementations, the system can generate the instructions to modify the position of the surgical robot by performing operations comprising generating the instructions to move the surgical robot according to movement of the patient.

At least one other aspect of the present disclosure is directed to a method of initiating collaborative control of a robot in response to detected conditions. The method can include controlling a position of a surgical robot in a surgical environment including a patient. The method can include detecting a collaborative control condition of the surgical robot based on a condition of the surgical environment. The method can include generating instructions to provide manual control of the surgical robot responsive to detecting the collaborative control condition.

In some implementations, the method can include accessing a three-dimensional (3D) point cloud corresponding to the patient in the surgical environment. In some implementations, detecting the collaborative control condition can include determining that one or more points of the 3D point cloud satisfy a movement condition. In some implementations, the method can include identifying a set of torque measurements captured from one or more torque sensors coupled to the patient. In some implementations, detecting the collaborative control condition further comprises determining that the set of torque measurements satisfy a patient movement condition. In some implementations, detecting the collaborative control condition can include detecting an error condition in an image-to-patient registration process. In some implementations, detecting the collaborative control condition can include receiving an interaction with a button corresponding to the collaborative control condition

In some implementations, controlling the position of the surgical robot comprises identifying one or more predetermined trajectories for an instrument to carry out a surgical procedure. In some implementations, identifying the one or more predetermined trajectories for the instrument comprises receiving, via user input, a selection of the one or more predetermined trajectories. In some implementations, controlling the position of the surgical robot comprises navigating the surgical robot along the one or more predetermined trajectories. In some implementations, controlling the position of the surgical robot comprises navigating the surgical robot in accordance with movement of the patient. In some implementations, detecting the collaborative control condition comprises identifying a set of torque measurements captured from one or more torque sensors coupled to the surgical robot. In some implementations, detecting the collaborative control condition comprises determining that the set of torque measurements satisfy a patient movement condition.

At least one other aspect of the present disclosure is directed to a method of initiating collaborative control of a robot in response to detected conditions. The system can include one or more processors coupled to a non-transitory memory. The system can control a position of a surgical robot in a surgical environment including a patient. The system can detect a collaborative control condition of the surgical robot based on a condition of the surgical environment. The system can generate instructions to provide manual control of the surgical robot responsive to detecting the collaborative control condition.

In some implementations, the system can access a three-dimensional (3D) point cloud corresponding the patient in the surgical environment. In some implementations, the system can detect the collaborative control condition by performing operations comprising determining that one or more points of the 3D point cloud satisfy a movement condition. In some implementations, the system can identify a set of torque measurements captured from one or more torque sensors coupled to the patient. In some implementations, the system can detect the collaborative control condition by performing operations comprising determining that the set of torque measurements satisfy a patient movement condition.

In some implementations, the system can detect the collaborative control condition by performing operations comprising detecting an error condition in an image-to-patient registration process. In some implementations, the system can detect the collaborative control condition by performing operations comprising receiving an interaction with a button corresponding to the collaborative control condition. In some implementations, the system can control the position of the surgical robot by performing operations comprising identifying one or more predetermined trajectories for an instrument to carry out a surgical procedure.

In some implementations, the system can identify the one or more predetermined trajectories for the instrument by performing operations comprising receiving, via user input, a selection of the one or more predetermined trajectories. In some implementations, the system can control the position of the surgical robot by performing operations comprising navigating the surgical robot along the one or more predetermined trajectories. In some implementations, the system can control the position of the surgical robot by performing operations comprising navigating the surgical robot in accordance with movement of the patient. In some implementations, the system can detect the collaborative control condition by performing operations comprising identifying a set of torque measurements captured from one or more torque sensors coupled to the surgical robot. In some implementations, the system can detect the collaborative control condition by performing operations comprising determining that the set of torque measurements satisfy a patient movement condition.

Various aspects relate generally to systems and methods for real-time multiple modality image alignment using three-dimensional (3D) image data, and can be implemented without markers and at sub-millimeter precision. 3D images, including scans such as CTs or MRIs, can be registered directly onto a subject, such as the body of a patient, that is captured in real-time using one or more capture devices. This allows for certain scan information, such as internal tissue information, to be displayed in real-time along with a point-cloud representation of the subject. This can be beneficial for surgical procedures that would otherwise utilize manual processes to orient instruments in the same frame of reference in a CT scan. Instruments can be tracked, instrument trajectories can be drawn, and targets can be highlighted on the scans. The present solution can provide real-time, sub-millimeter registration for various applications such as aligning depth capture information with medical scans (e.g., for surgical navigation), aligning depth capture information with CAD models (e.g., for manufacturing and troubleshooting), aligning and fusing multiple medical image modalities (e.g., MRI and CT; CT and 3D ultrasound; MRI and 3D ultrasound), aligning multiple CAD models (e.g., to find differences between models), and fusing depth capture data from multiple image capture devices).

The present solution can be implemented for image-guided procedures in various settings, including operating rooms, outpatient settings, CT suites, ICUs, and emergency rooms. The present solution can be used for neurosurgery applications such as CSF-diversion procedures, such as external ventricular placements and VP shunt placements; brain tumor resections and biopsies; and electrode placements. The present solution can be used for interventional radiology, such as for abdominal and lung biopsies, ablations, aspirations, and drainages. The present solution can be used for orthopedic surgery, such as for spinal fusion procedures. The present solution can be used for non-invasive surgical navigation, such as transcranial magnetic stimulation (TMS) and focused ultrasound (FUS) by combining the image guidance of the present solution with surgical instruments. The present solution allows for robotic control of the surgical instrument for non-invasive cranial procedures and utilizes the real-time registration to target highlighted locations of interest.

At least one aspect of the present disclosure relates to a method of delivering procedure to a location of interest through a surgical instrument. The method can be performed, by one or more processors of a data processing system. The method can include, by one or more processors, registering a 3D medical image that is positioned relative to a frame of reference. The method can include receiving tracking data of the surgical instrument being used to perform the procedure. The method can include determining a relative location of the surgical instrument to the location of interest within the frame of reference that is related to a first point cloud and the 3D medical image. The method can include tracking for target movement and adjusting the surgical instrument to remain aligned with the location of interest. The method can include delivering procedure to the location of interest through the surgical instrument. The method can include receiving a threshold for the procedure and a parameter detected during the procedure. The method can include causing the surgical instrument to terminate the procedure which is in response to the parameter satisfying the threshold. In some implementations of the method, the location of interest is on a surface of a head of a subject.

In some implementations of the method, transforming the tracking data from the surgical instrument can include using the first reference frame to generate a transformed tracking data. In some implementations of the method, rendering the transformed tracking data can be included within the render of the first point cloud and the 3D medical image.

In some implementations of the method, generating movement instructions for the surgical instrument can be based on the first point cloud, the 3D medical image, and the location of interest. In some implementations of the method, transmitting the movement instructions can include the surgical instrument. In some implementations of the method, displaying a highlighted region for the location of interest can be included within a render of the 3D medical image and the first point cloud. In some implementations of the method, determining the distance of the subjected represented in the 3D medical image from a capture device can be responsible at least in part for generating the first point cloud.

In some implementations of the method, causing the surgical instrument to terminate energy emission can include the location of interest not being within the frame of reference. In some implementations of the method, causing the surgical instrument to terminate energy emission can include the target movement exceeding the surgical instrument movement for procedure to the location of interest.

In some implementations of the method, allowing the surgical instrument to contact the target can include being responsive to target movement by combining the registered 3D medical image and the first point cloud with torque sensing. In some implementations of the method, receiving the tracking data from the surgical instrument can include applying a force to keep the surgical instrument in contact with the surface. In some implementations of the method, transforming the tracking data from the surgical can be relative to detected target movement and can also include maintaining the force originally applied to the surface.

At least one other aspect of the present disclosure relates to a system that delivers procedure to a location of interest through a surgical instrument. The system can register, by one or more processors, a 3D medical image positioned relative to a frame of reference. The system can receive, by one or more processors, tracking data of a surgical instrument and determine a relative location of the surgical instrument to the location of interest within the frame of reference related to a first point cloud and the 3D medical image. The system can track, by one or more processors based on the relative location, target movement and adjust the surgical instrument to remain aligned with the location of interest. The system can deliver, by one or more processors, procedure to the location of instrument through the surgical instrument and receive a threshold for the procedure and a parameter detected during the procedure. The system can cause, by one or more processors, the surgical instrument to terminate the procedure which is responsive to the parameter satisfying the threshold. In some implementations of the system, the location of interest can be on a surface of a head of a subject.

In some implementations of the system, the system can transform the tracking data from the surgical instrument to the first reference frame to generate a transformed tracking data. In some implementations of the system, the system can render the transformed tracking data within the render of the first point cloud and the 3D medical image.

In some implementations of the system, the system can generate movement instructions for the surgical instrument based on the first point cloud, the 3D medical image, and the location of interest. In some implementations of the system, the system can transmit the movement instructions to the surgical instrument. In some implementations of the system, the system can display a highlighted region within a render of the 3D medical image and the first point cloud that corresponds to the location of interest. In some implementations of the system, the system can determine a distance of the subject and be represented in the 3D medical image from a capture device responsible at least in part for generating the first point cloud.

In some implementations of the system, the system can cause the surgical instrument to terminate energy emission if the location of interest is not within the frame of reference. In some implementations of the system, the system can cause the surgical instrument to terminate energy emission if the target movement exceeds the surgical instrument movement for procedure to the location of interest.

In some implementations of the system, the system can allow the surgical instrument to contact the target and can also be responsive to the target movement. In some implementations of the system, the system can combine the registered 3D medical image and the first point cloud with torque sensing. In some implementations of the system, the system can receive the tracking data from the surgical instrument and apply a force to keep the surgical instrument in contact with the surface. In some implementations of the system, the system can transform the tracking data from the surgical instrument relative to detected target movement and maintain the force originally applied to the surface.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form. For example, by appropriate computer programs, which can be carried on appropriate carrier media (computer readable media), which can be tangible carrier media (e.g. disks) or intangible carrier media (e.g. communications signals). Aspects can also be implemented using suitable apparatus, which can take the form of programmable computers running computer programs arranged to implement the aspect. As used in the specification and in the claims, the singular form of ‘a’, ‘an’, and ‘the’ include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing. In the drawings:

FIGS. 1A and 1B show perspective views of an example image processing system, in accordance with one or more implementations;

FIG. 2 is a block diagram of an image processing system capable of monitoring a position of a patient and a robot, in accordance with one or more implementations;

FIG. 3 is a perspective view of an example robot control system, in accordance with one or more implementations;

FIG. 4 is a block diagram of a robot control system capable of controlling a surgical robot based on patient tracking, in accordance with one or more implementations;

FIG. 5 is a flow diagram of an example method of controlling a surgical robot based on patient tracking, in accordance with one or more implementations;

FIG. 6 is a block diagram of a robot control system capable of controlling a surgical robot based on torque sensing techniques, in accordance with one or more implementations; and

FIG. 7 is a flow diagram of an example method of controlling a surgical robot based on torque sensing techniques, in accordance with one or more implementations;

FIG. 8 is a block diagram of a robot control system capable of initiating collaborative control of surgical robots in response to detected conditions, in accordance with one or more implementations;

FIG. 9 is a flow diagram of an example method of initiating collaborative control of surgical robots in response to detected conditions, in accordance with one or more implementations;

FIG. 10 is a block diagram of an image processing system including a surgical instrument, in accordance with one or more implementations;

FIG. 11 is a flow diagram of a method for real-time non-invasive surgical navigation, in accordance with one or more implementations; and

FIGS. 12A and 12B are block diagrams of an example computing environment, in accordance with one or more implementations.

### DETAILED DESCRIPTION

Below are detailed descriptions of various concepts related to, and implementations of, techniques, approaches, methods, apparatuses, and systems for managing surgical tools having integrated display devices. The various concepts introduced above and discussed in greater detail below can be implemented in any of numerous ways, as the described concepts are not limited to any particular manner of implementation. Examples of specific implementations and applications are provided primarily for illustrative purposes.

For purposes of reading the description of the various embodiments below, the following descriptions of the sections of the Specification and their respective contents can be helpful:
Section A describes hardware components that may implement the robot control techniques described herein;
Section B describes techniques for controlling a surgical robot based on patient tracking techniques; and
Section C describes techniques for controlling a surgical robot based on torque sensing techniques;
Section D describes techniques for initiating collaborative control of surgical robots in response to detected conditions;
Section E describes techniques for real-time non-invasive navigation; and
Section F describes a computing environment which can be useful for practicing implementations described herein.

### A. Hardware Components and System Architecture

The image tracking, torque sensing, and robot control techniques described herein can take place in real-time in a surgical environment, for example, during a cranial surgical procedure. Prior to discussing in detail the particular techniques for surgical robot control based on image registration, surgical robot control based on torque sensing, and initiating collaborative control response to detected conditions in the surgical environment, it is helpful to describe the particular components disposed within the surgical environment with which such techniques may operate.

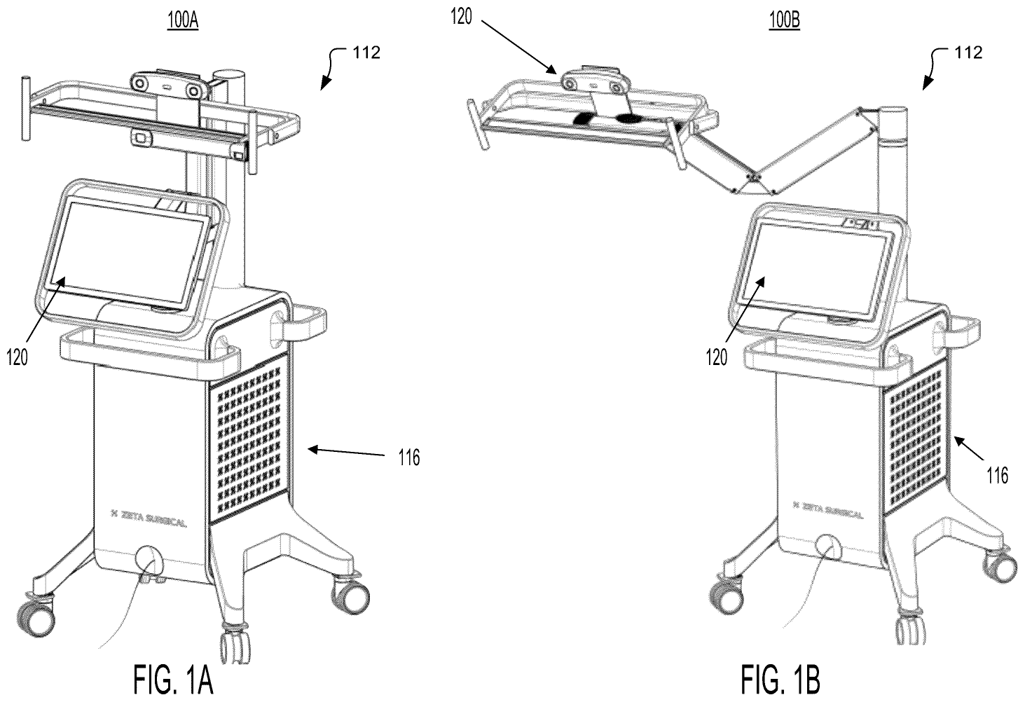

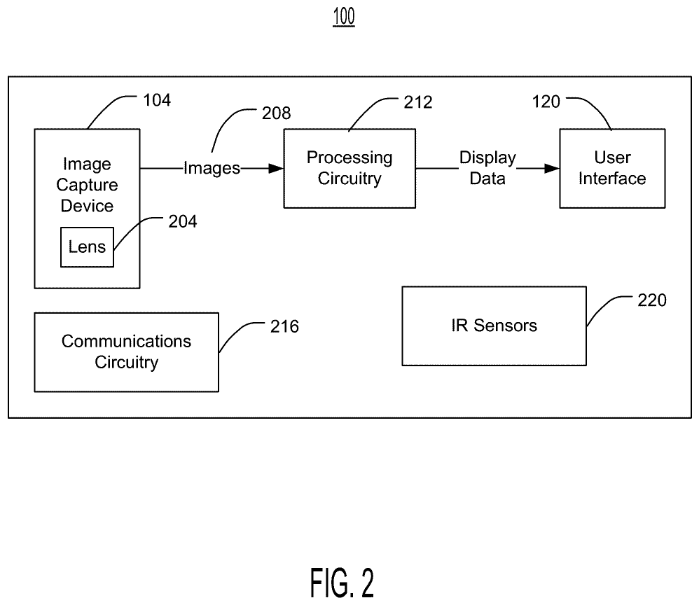

FIGS. 1A, 1B and 2 depict an image processing system 100. The image processing system 100 can include a one or more image capture devices 104, such as three-dimensional (3D) cameras. The cameras can be visible light cameras (e.g., color or black and white), infrared cameras (e.g., the IR sensors 220, etc.), or combinations thereof. Each image capture device 104 can include one or more lenses 204. In some embodiments, the image capture device 104 can include a camera for each lens 204. The image capture devices 104 can be selected or designed to be a predetermined resolution and/or have a predetermined field of view. The image capture devices 104 can have a resolution and field of view for detecting and tracking objects. The image capture devices 104 can have pan, tilt, or zoom mechanisms. The image capture device 104 can have a pose corresponding to a position and orientation of the image capture device 104. The image capture device 104 can be a depth camera. The image capture device 104 can be the KINECT manufactured by MICROSOFT CORPORATION.

Light of an image to be captured by the image capture device 104 be received through the one or more lenses 204. The image capture devices 104 can include sensor circuitry, including but not limited to charge-coupled device (CCD) or complementary metal-oxide-semiconductor (CMOS) circuitry, which can detect the light received via the one or more lenses 204 and generate images 208 based on the received light.

The image capture devices 104 can provide images 208 to processing circuitry 212, for example via a communications bus. The image capture devices 104 can provide the images 208 with a corresponding timestamp, which can facilitate synchronization of the images 208 when image processing is executed on the images 208. The image capture devices 104 can output 3D images (e.g., images having depth information). The images 208 can include a plurality of pixels, each pixel assigned spatial position data (e.g., horizontal, vertical, and depth data), intensity or brightness data, and/or color data. When captured in a surgical environment that includes, for example, a surgical robot operating on a patient, the images 208 can include pixels that represent the portions of the surgical robot, such as the tool end of the surgical robot, or markers positioned on the surgical robot, among others. In implementations where the image capture devices 104 are 3D cameras, the surgical robot and the patient can be mapped to corresponding 3D point clouds in the reference frame of the image capture devices 104. The 3D point clouds can be stored, for example, in the memory of the processing circuitry 212, and provided to the robot controller systems described herein. In some implementations, the processing circuitry 212 can perform image-to-patient registration (e.g., registration between a CT image of the patient and the 3D point cloud representing the patient), in addition to tracking the 3D point clouds corresponding to the patient.

Each image capture device 104 can be coupled with the platform 112, such as via one or more arms or other supporting structures, and can be communicatively coupled to the processing circuitry 212. The platform 112 can be a cart that can include wheels for movement and various support surfaces for supporting devices to be used with the platform 112. In some implementations, the platform 112 is a fixed structure without wheels, such as a table. In some implementations, the components coupled to the platform 112 can be modular and removable, such that they can be replaced with other tracking devices or computing devices as needs arise.

The platform 112 can support processing hardware 116 (which is described in further detail below in conjunction with FIG. 2) that includes at least a portion of the processing circuitry 212, as well as the user interface 120. The user interface 120 can be any kind of display or screen as described herein, and can be used to display, for example, a three-dimensional rendering of the environment captured by the image capture devices 104. Images 208 can be processed by processing circuitry 212 for presentation via user interface 120. As described above, the images 208 can include representations of the patient or the surgical tool, which are positioned within the surgical environment captured by the image capture devices 104. In some implementations, the processing circuitry 212 can utilize one or more image classification techniques (e.g., deep neural networks, light detection, color detection, etc.) to determine the location (e.g., pixel location, 3D point location, etc.) of the surgical robot, surgical tools, or the patient, as described herein.

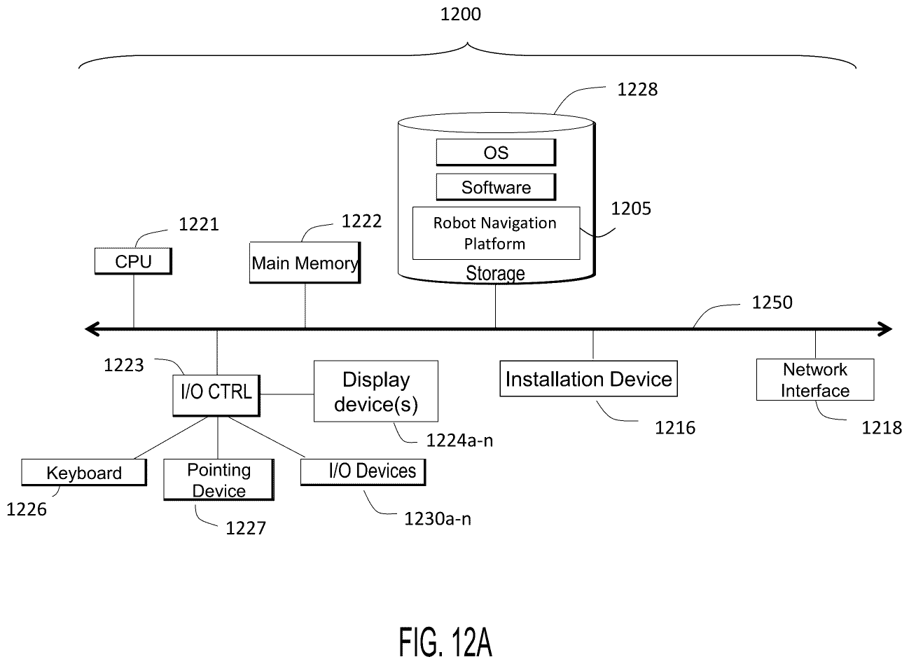

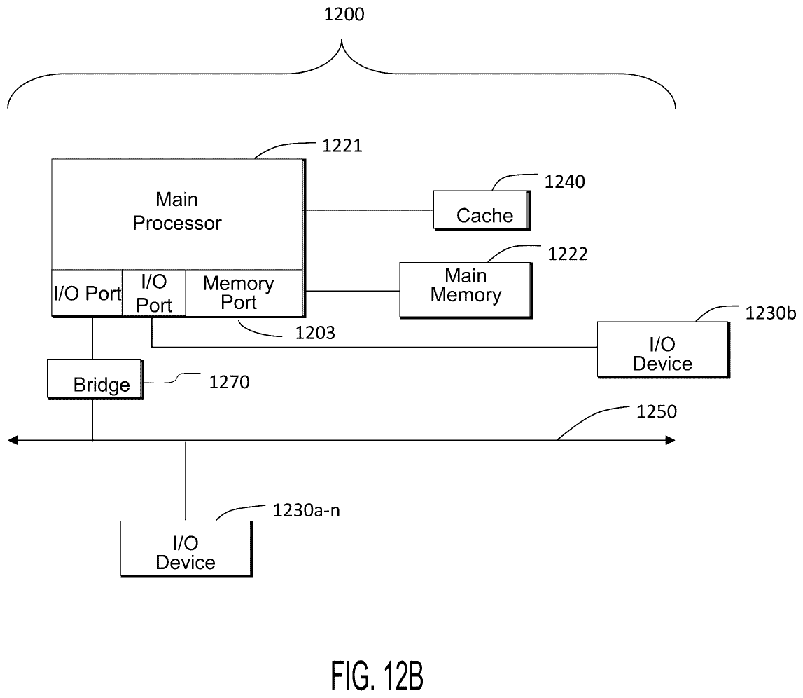

Processing circuitry 212 can incorporate features of computing device 1000 described with reference to FIGS. 12A and 12B. For example, processing circuitry 212 can include processor(s) and memory. The processor can be implemented as a specific purpose processor, an application specific integrated circuit (ASIC), one or more field programmable gate arrays (FPGAs), a group of processing components, or other suitable electronic processing components. The memory is one or more devices (e.g., RAM, ROM, flash memory, hard disk storage, etc.) for storing data and computer code for completing and facilitating the various user or client processes, layers, and modules described in the present disclosure. The memory can be or include volatile memory or non-volatile memory and can include database components, object code components, script components, or any other type of information structure for supporting the various activities and information structures of the features described herein. The memory can be communicably connected to the processor, and can include computer code or instruction modules for executing one or more operations described herein. The memory can include various circuits, software engines, and/or modules that cause the processor to execute the operations described herein.

Some portions of processing circuitry 212 can be provided by one or more devices remote from platform 112. For example, one or more servers, cloud computing systems, or mobile devices (e.g., as described with reference to FIGS. 12A and 12B), can be used to perform various image processing techniques described herein.

The image processing system 100 can include communications circuitry 216. The communications circuitry 216 can implement features of computing device 1000 described with reference to FIGS. 12A and 12B, such as network interface 1218. The communications circuitry 216 can be used, for example, to communicate information relating to the position of the 3D point cloud corresponding to a patient in the surgical environment, which can be used in the processing components described herein to navigate a surgical robot. In some implementations, the communications circuitry 216 can be used to communicate with the robot control systems 405, 605, or 805. In some implementations, the image processing system 100 can implement one or more of the functionalities of any of the robot control systems 405, 605, or 805 described herein. The communications circuitry 216 can be any type of input/output interface that is capable of communication information between the image processing system 100 (or the components thereof) and one or more components, devices, or systems, including any components, devices, or systems described herein.

The image processing system 100 can include one or more infrared (IR) sensors 220. The IR sensors 220 can detect IR signals from various devices in an environment around the image processing system 100. For example, the IR sensors 220 can be used to detect IR signals from IR emitters that can be coupled to tracked features in the surgical environment, such as a portion of the patient, a portion of a surgical robot, or a tool-end coupled to the surgical robot, among others. The IR sensors 220 can be communicatively coupled to the other components of the image processing system 100, such that the components of the image processing system 100 can utilize the IR signals in appropriate operations in the techniques described herein.

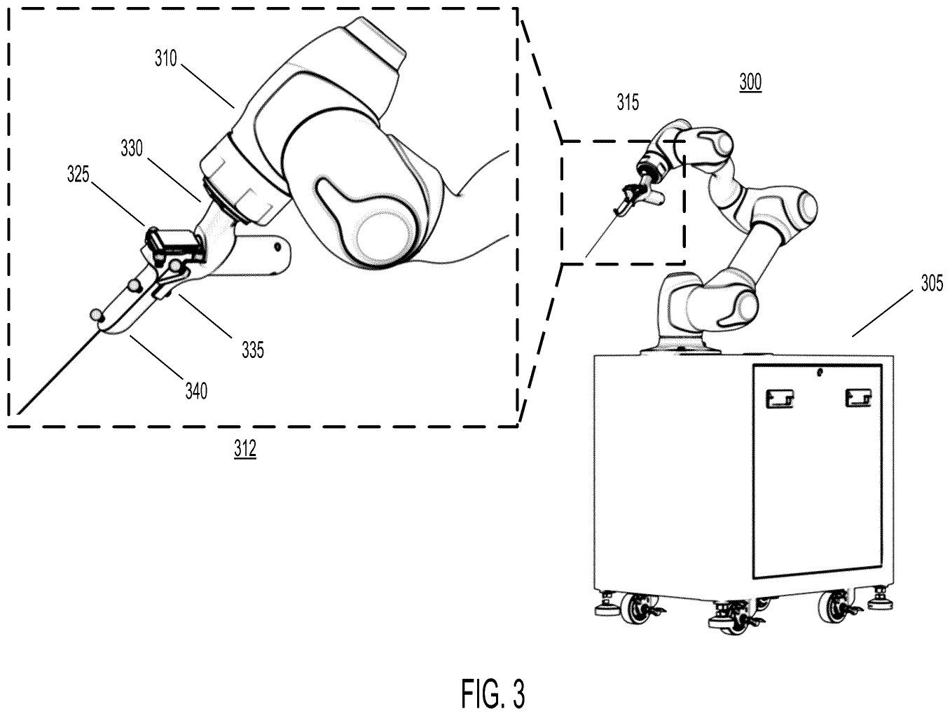

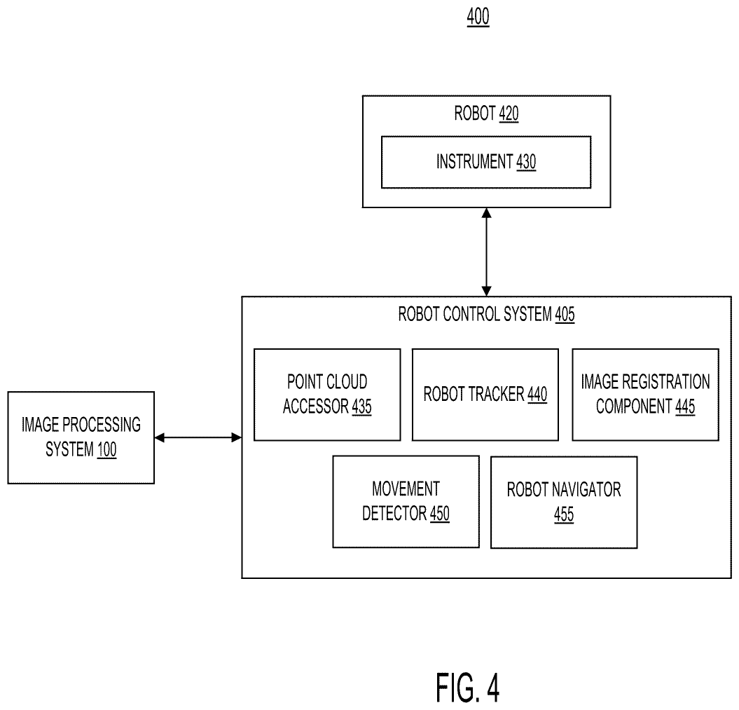

Referring now to FIG. 3, depicted is a robot system 300 of an example robot control system, in accordance with one or more implementations. The robot system 300 can include a cart 305, which may include one or more of the robot control systems 405, 605, or 805 as described herein in connection with FIGS. 4, 6, and 8. Mounted on the cart is a robotic arm 310, which can be in communication with or controlled by the robot control systems 405, 605, or 805. A close-up of the tracked end effector 315 is shown in the close-up view 312. As shown in the close-up view 312, the tracked end effector 315 of the robotic arm 310 can include a screen 325, an instrument holder 330, one or more buttons 335, and a tracked instrument 340 (e.g., shown here as a catheter guide including one or more markers) that is connected to the instrument holder 330. The instrument holder 330 can be a universal attachment or connector on the robot arm 310 for tracked instruments 340. For example, the instrument hold can allow the robot arm 310 to be used with any type tracked instrument 340.

The cart 305 can be similar to, and can include any of the structure or functionality of, the platform 112. The cart 305 can include wheels for movement and can support the other devices shown in the robot system 300. The robotic arm 310 can be any type of robotic arm that can be navigated in 3D space in accordance with instructions provided by the robotic control systems 405, 605, and 805 described herein. The robotic arm 310 can be controlled automatically according to a predetermined pathway in 3D space, or can be controlled in a partially automated state in which a surgeon or other medical professional can move the robotic arm 310 within predetermined boundaries established through software. In some implementations, in response to various conditions described herein, the robotic arm 310 can enter a “collaborative mode,” in which manual control over the position and orientation of the robotic arm 310 (e.g., tracked instrument 340, etc.) are provided to the surgeon. Such techniques are described in greater detail herein in connection with Section D. In some implementations, the robotic arm 310 can be an M0609 robotic arm manufactured by DOOSAN ROBOTICS. The robotic arm 310 can be a computer-controlled electromechanical multi-jointed arm.

The tracked end effector 315 includes the screen 325, the instrument holder 330, and the tracked instrument 340. In some implementations, the tracked end effector 315 includes one or more buttons 335, which when interacted with can allow the surgeon to navigate through different points or sequences of 3D pathways to perform surgical operations. In some implementations, one or more of the buttons 335 can initiate collaborative control mode, allowing the surgeon to have complete control over the position and orientation of the robotic arm 310. The robotic arm 310 can receive instructions, for example, via a communications interface from the computing devices described herein, that include movement instructions. For example, the movement instructions can be instructions that cause the robotic arm 310 to modify the position of the tracked instrument (e.g., by actuating one or more joints according to its internal programming, etc.) to a desired position or orientation within a surgical environment. In addition, the surgical robot may transmit messages to the computing devices described herein to provide information related to a status of the robotic arm 310 (e.g., whether the robotic arm is in an automatic navigation mode, whether the robotic arm is in collaborative mode, etc.).

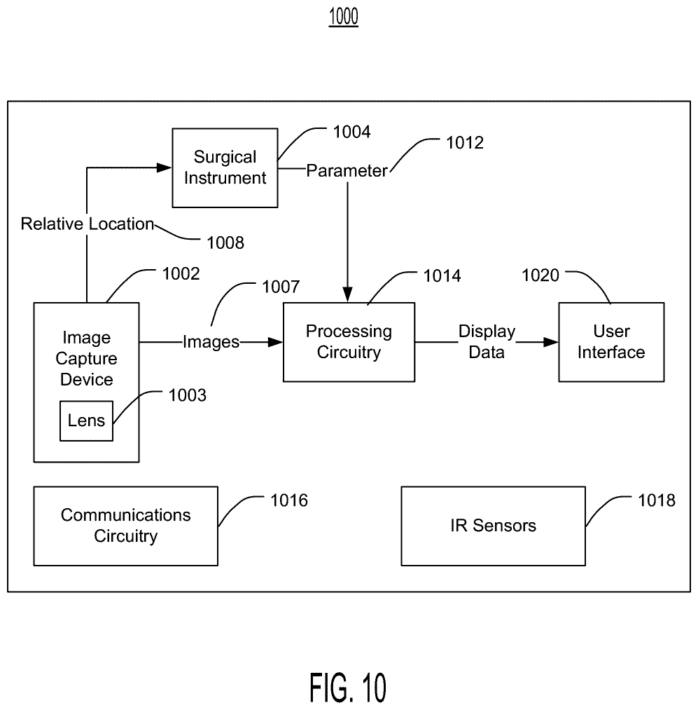

The tracked instrument 340 can include any type of surgical instrument, and is shown here as a catheter guide that can be used in neurosurgical operations. In some implementations, the tracked instrument 340 can be coupled to one or more markers, allowing the position and orientation of the tracked instrument 340 within the surgical environment to be determined. For example, in some implementations, the processing circuitry 212 of the image processing system 100, or the image processing system 1000 of FIG. 10, can track the position of the instrument with respect to the 3D point cloud representing the patient. In some implementations, the tracking markers can be coupled to the robotic arm 310, for example, to track the position of the robotic arm 310 or its various joints.

The systems and methods described herein can be utilized at the bedside in a surgical environment. For example, both the platform 112, including the image processing system 100, and the robot system 300, which can include any of the robot control systems 405, 605, or 805, or the image processing system 1000, as described herein, can be positioned within the surgical environment including a patient. The image processing system 100 can perform image-to-patient registration processes to align 3D images from computed tomography (CT) scans or from magnetic resonance imaging (MRI) scans with the 3D point cloud of patient captured by the image capture devices 104. In addition, the position of the patient's face can be determined based on the position of the 3D point cloud representing the patient. The processing circuitry 212 may also capture and track the position of the robotic arm 310 or the tracked instrument 340 in the same frame of reference as the 3D point cloud representing the patient, allowing the processing circuitry 212 to determine the distance of the tracked instrument 340 from the patient or from predetermined surgical pathways. The techniques for updating the position of the surgical robot 310 based on various attributes of the surgical environment (e.g., detected patient movement, torque sensing, etc.) are described in greater detail in the following sections.

### B. Controlling a Surgical Robot Based On Patient Tracking Techniques

The systems and methods described herein provide various techniques for controlling a surgical robot in a surgical environment. In particular, the techniques described herein provide improved movement tracking and adjustment for surgical robots based on real-time 3D images of a patient in a surgical environment. Using an image processing system 100 to perform patient tracking techniques, the systems and methods described herein can determine precise patient movement in near real-time, while navigating a surgical robot along a predetermined path in a surgical environment. The surgical robot can be controlled such that a tracked instrument (e.g., the tracked instrument 340) aligns with the predetermined pathway or target. For example, the target can be an intracranial target. The location of the intracranial target can be determined, for example, based on the location of a point of interest in a CT scan image or an MRI scan image that is aligned with the real-time 3D image using image-to-patient registration techniques. When patient movement is detected, the trajectory or position of the tracked instrument used in the surgical procedure can be adjusted to maintain this alignment. The systems and methods described herein improve surgical robot navigation technology by enabling real-time correction of surgical pathways using real-time patient tracking. The technology described herein improves patient safety during surgical procedures.

Referring now to FIG. 4, depicted is an example system 400 for controlling a surgical robot (e.g., the robot 310, etc.) based on patient tracking techniques, in accordance with one or more implementations. The system 400 can include at least one robot control system 405, at least one robot 420, and at least one image processing system 100. The robot control system 405 can include at least one point cloud accessor 435, at least one robot tracker 440, at least one image registration component 445, at least one movement detector 450, and at least one robot navigator 455. The robot 420 can include an instrument 430.

Each of the components (e.g., the robot control system 405, the image processing system 100, the robot 420, etc.) of the system 400 can be implemented using the hardware components or a combination of software with the hardware components of a computing system (e.g., computing system 1000, any other computing system described herein, etc.) detailed herein in conjunction with FIGS. 12A and 12B. Each of the components of the robot control system 405 (e.g., the point cloud accessor 435, the robot tracker 440, the image registration component 445, the movement detector 450, the robot navigator 455, etc.) can perform the functionalities detailed herein. It should be understood that although the imaging processing system 100 and the robot control system 405 are depicted as separate systems, that the robot control system 405 may be a part of the image processing system 100 (e.g., implemented at least in part by the processing circuitry 212, etc.) or vice versa (e.g., the processing circuitry 212 of the image processing system 100 implemented on one or more processors of the robot control system 405). Similarly, the robot control system 405 may be implemented with or may include the image processing system 1000 described in connection with FIG. 10, or vice versa. In implementations where the image processing system 100 and the robot control system 405 are implemented as separate computing systems, the image processing system 100 and the robot control system 405 can exchange information via a communications interface, as described herein. Likewise, the robot control system 405 and the robot 420 can communicate via one or more communications interfaces. The robot control system 405 can communicate any generated instructions to the robot 420 for execution.

The robot control system 405 can be, or form a part of, the image processing system 100 described herein in conjunction with FIGS. 1A, 1B, and 2, and can perform any of the functionalities of the image processing system 100 as described herein. The robot control system 405 can include at least one processor and a memory (e.g., a processing circuit). The memory can store processor-executable instructions that, when executed by processor, cause the processor to perform one or more of the operations described herein. The processor can include a microprocessor, an ASIC, an FPGA, a graphics processing unit (GPU), etc., or combinations thereof. The memory can include, but is not limited to, electronic, optical, magnetic, or any other storage or transmission device capable of providing the processor with program instructions. The memory can further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions can include code from any suitable computer programming language. The robot control system 405 can include one or more computing devices or servers that can perform various functions as described herein. The robot control system 405 can include any or all of the components and perform any or all of the functions of the computer system 1000 described herein in connection with FIGS. 12A and 12B.

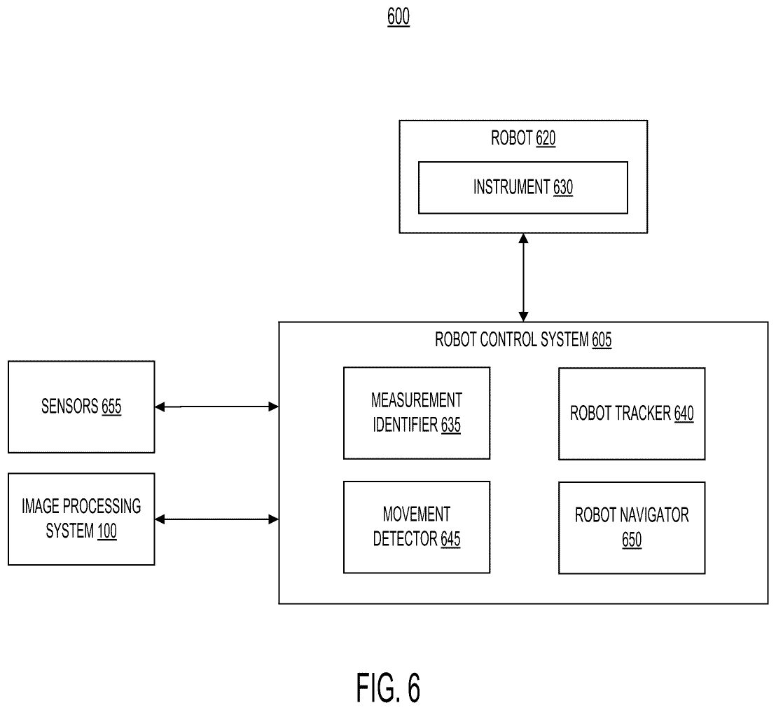

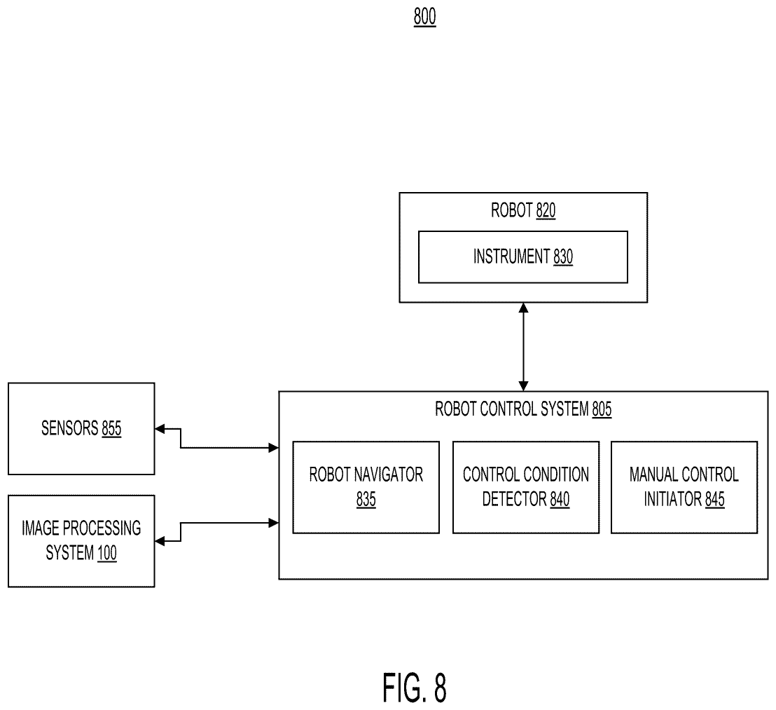

The robot 420 can be, or can include any of the functions or structure of, the robotic arm 310 described herein above in connection with FIG. 3. In some implementations, the robot 420 can be a different type of surgical robot that is capable of maneuvering a surgical instrument in a surgical environment. The robot 420 can include a computing device that executes instructions to move the robot to a desired position or orientation in the surgical environment. The robot control system 405 (or the components thereof) can generate instructions for the robot 420 that cause the robot 420 to change its position, orientation, or status (e.g., automatic or collaborative, etc.). The robot 420 can operate in an automatic mode, in which the position of the robot 420 (and the instrument 430 coupled thereto) is controlled by software (e.g., the robot navigator 455 described in connection with FIG. 4, the robot navigator 650 described in connection with FIG. 6, the robot navigator 835 described in connection with FIG. 8, any other components of the robot control systems 405, 605, or 805 as described herein, etc.). If the robot 420 is in the appropriate status, the robot 420 can operate in a collaborative mode, in which the robot 420 can be controlled entirely, or partially, by manual input of a surgeon. For example, the surgeon may hold on to portions of the robot to position the robot 420 or the instrument 430 towards a desired target.

In some implementations, and as described herein above in connection with FIG. 4, the robot 420 can include a display that is positioned over the patient in the surgical environment, such that the surgeon can view information about the patient (e.g., a close up view of the surgical site with annotations, information relating to a target or other target positions of the surgical procedure, etc.) as the surgical procedure is performed. In some implementations, the robot can include a capture device, similar to one of the image capture devices 104 described herein above. The images 208 captured by the image capture device coupled to the robot 420 can be displayed on the display coupled to the robot 420 and positioned over the surgical site.

The instrument 430 can be any type of instrument that may be used with in a surgical environment for surgical procedures on a patient. The instrument 430 can be coupled to the robot 420, such that the robot 420 can control the position and orientation of the instrument 430 in space. The instrument 430 can be, for example, a drilling tool, a cannula needle, a biopsy needle, a catheter device, or any other type of surgical instrument. The instrument 430 can be, and can include any of the structure and functionality of, the tracked instrument 340. For example, the instrument 430 (or the bracket that couples the instrument 430 to the robot 420) can be coupled to one or more tracking indicators. The tracking indicators can be, for example, IR light-emitting diodes (LEDs), LEDs that emit color in the visual spectrum, tracking balls colored with a predetermined color or having a predetermined, detectable shape, or other tracking features, such as QR codes. The tracking indicators can be positioned on predetermined places on the instrument 430 or the robot 420, and can form a matrix or array of sensors that, when detected by a computing device (e.g., the image processing system 100, the robot control system 405, etc.), can be used to determine a position and orientation of the instrument 430 or the robot 420. In some implementations, the instrument 430 can include or be coupled to one or more position sensors, such as accelerometers, gyroscopes, or inertial measurement units (IMUs), among others.

The point cloud accessor 435 can access a three-dimensional (3D) point cloud corresponding to a surgical environment and a patient. The 3D point cloud can have a frame of reference that corresponds to a surgical environment. As described herein above, the image processing system 100 can utilize one or more image capture devices 104, which can be 3D cameras, to capture a real-time (or near real-time) 3D image of a patient during a surgical procedure. The 3D point cloud can correspond, for example, to the head of the patient, the body of the patient, or to any other portion of the patient on which surgery can be performed. In some implementations, the image capture devices 104 can be positioned in the surgical environment to capture an image of the patient's face. In some implementations, the point cloud accessor 435 can apply an image segmentation model to the 3D point cloud captured by the image capture devices 104. In some implementations, the point cloud accessor 435 can receive the point cloud from the processing circuitry 212 of the image processing system 100 of FIGS. 1 and 2, or the processing circuitry 1014 of the image processing system 1000 of FIG. 10, for example, via one or more communication interfaces. In some implementations, the point cloud accessor 435 can capture an indication of a global environment (e.g., a static point in the surgical environment from which the 3D point cloud corresponding to the patient may be accessed, etc.). The point cloud accessor 435 can receive 3D images including point clouds representing the patient iteratively, for example, at a predetermined framerate of the image capture devices 104. The point cloud accessor 435 can access or otherwise retrieve the 3D point clouds representing the user and store the 3D point clouds in the memory of the robot control system 405, such that they can be accessed by the other components of the robot control system. The 3D point clouds can be stored in association with a respective timestamp, such that the position and orientation of the patient can be determined in real-time or near real-time.

In addition to tracking the position of the patient, the robot tracker 440 can determine a position of a surgical robot within the same frame of reference as the 3D point cloud. As described herein above, the image capture devices 104 can capture images of a patient in a surgical environment. The image capture devices 104 can have a predetermined pose within the surgical environment relative to other sensors, such as the IR sensors 220. As described herein above, the IR sensors 220 can be IR cameras that capture IR wavelengths of light. The robot tracker 440 can determine the position of the robot by utilizing one or more tracking techniques, such as IR tracking techniques, to determine the position of the robot 420. The robot 420 can include one or more markers or indicators on its surface, such as IR indicators. The robot tracker 440 can utilize the IR sensors 220 to determine the relative position of the robot. In some implementations, the robot tracker 440 can determine the orientation (e.g., the pose) of the robot 420 by performing similar techniques. Because the sensors used to track the position of the robot 420 are a known distance from the image capture devices 104, the position of the markers detected by the IR sensors 220 can be mapped to the same frame of reference as the 3D point cloud captured by the image capture devices 104.

In some implementations, the image capture devices 104 may be used to determine the position of the robot 420. For example, the robot 420 can include one or more graphical indicators, such as bright distinct colors, patterns or QR codes, among others. In addition to capturing 3D point clouds of the patient, the image capture devices 104 can capture images of the surgical environment, and the robot tracker 440 can perform image analysis techniques to determine the position of the surgical robot in the images 208 based on the detected positions of the indicators in the images 208. The position and orientation of the surgical robot can be computed periodically, for example, in real-time or near real-time, enabling the robot tracker 440 to track the movement of the robot 420 over time. In some implementations, the robot tracker 440 can perform a calibration procedure, for example, to establish the unified frame of reference between the 3D point clouds and the indicators coupled to the surgical robot. The calibration procedure can include identifying the position of the robot 420 with respect to global indicators positioned in the surgical environment. The robot tracker 440 can store the position of the robot 420 in the memory of the robot tracker 440 such that the real-time or near real-time position of the robot 420 can be accessed by other components of the robot control system 405. The calibration procedure can include using predetermined markers or patterns in the surgical environment (e.g., a chessboard pattern, etc.) to calibrate the pose (e.g., the position and orientation) of the robot 420 with respect to the patient.

Tracking the position of the robot 420 can include tracking the position of the instrument 430. As described herein above, the instrument 430 can include its own indicators that are coupled to the instrument 430 or a bracket/connector coupling the instrument 430 to the robot 420. An example of indicators coupled to the instrument 430 is shown as the tracked instrument 340 in the close-up view 312 depicted in FIG. 3. The robot tracker 440 can track the position and orientation of the instrument 430 as well as the robot in real-time or near real-time using techniques similar to those described above. The position and orientation of the instrument 430 determined by the robot tracker 440 can be stored in the memory of the robot control system 405. In some implementations, the robot tracker 440 can store the position and orientation of the instrument 430 in association with a respective timestamp.

The image registration component 445 can perform an image-to-patient registration technique to align a 3D image of the patient, which may include indicators of potential targets, and the 3D point cloud captured from the patient. For example, for inter-cranial operations, a target may be a biopsy region with in the patient's skull. A 3D image, such as a CT scan of the patient's head, can include both a 3D representation of the patient's face and an indication of the biopsy region within the patient's brain. By registering the 3D image of the patient with the real-time 3D point cloud of the patient in the surgical environment, the image registration component 445 can map the 3D image of the patient, and any target indicators, into the same frame of reference as both the 3D point cloud, the tracked position of the robot 420, and the tracked position of the instrument 430. Doing so allows the robot tracker 440 to track the position of the instrument 430 with respect to both the patient and the target regions indicated in the 3D image of the patient. To register the 3D image to the 3D point cloud captured by the image capture devices 104, the image registration component 445 can perform an iterative fitting process, such as a random-sample consensus (RANSAC) algorithm and an iterative closest point (ICP) algorithm. The image registration component 445 can continuously register the 3D image to the 3D point cloud of the patient. If registration fails (e.g., the fitting algorithm fails to fit the 3D image to the 3D point cloud within a predetermined error threshold, etc.), the image registration component 445 can generate a signal indicating the failure.

As described herein above, the point cloud accessor 435 can continuously (e.g., each time an image is captured by the image capture devices 104, etc.) track the position of a patient over time in a surgical environment. The movement detector 450 can detect a change in a position of the patient by comparing the positions of the 3D point cloud over time. For example, when the point cloud accessor 435 receives or accesses a new 3D point cloud based on a new image captured by the image capture devices 104, the point cloud accessor 435 can store positions of each point in the 3D point cloud in the memory of the robot control system 405, for example, in one or more data structures. The data structures can be timestamped, or an index or other indication of order can otherwise be encoded in the data structures such that the order in which the 3D point clouds were captured can be determined by the components of the robot control system 405. The point cloud accessor 435 may store new 3D point clouds representing the patient, for example, in a rolling queue, such that a predetermined number of recent frames captured by the image capture devices 104 are stored in the memory of the robot control system 405.

The movement detector 450 can access the 3D point clouds stored in the memory of the robot control system 405, to determine an amount of patient movement in the surgical environment. One improvement of the systems and methods described herein when used during surgical procedures is that the techniques provide accurate patient tracking when the patient is not restrained. Certain surgical procedures, including some inter-cranial procedures, can be performed without securing the patient to a surgical operating table or apparatus. The movement detector 450 can accurately detect patient movement in such scenarios, and generate signals for the components of the robot control system 405 to adjust the position of the robot 420 to accommodate for patient movements. The movement detector 450 can compare previous positions of the 3D point cloud representing the patient to the positions of current or new 3D point clouds captured by the image capture devices 104. In some implementations, the movement detector 450 can maintain two sets of 3D point clouds: one from a previously captured frame and another from a currently captured (e.g., most-recently captured) frame. The comparison can be a distance (e.g., a Euclidean distance, etc.) in the frame of reference of the 3D point clouds.

The movement detector 450 can perform iterative computations to determine which 3D points in a first 3D point cloud (e.g., the previous frame) correspond to 3D points in a second 3D point cloud (e.g., the current frame). For example, the movement detector 450 can perform an iterative ICP algorithm or a RANSAC fitting technique to approximate which points in the current frame correspond to the points in the previous frame. Distances between the corresponding points can then be determined. In some implementations, to improve computational performance, the movement detector 450 can compare a subset of the points (e.g., by performing a down-sampling technique on the point clouds, etc.). For example, after finding the point correspondences, the movement detector 450 can select a subset of the matching point pairs between each point cloud to compare (e.g., determine a distance between in space, etc.). In some implementations, to determine the movement of the patient in the surgical environment, the movement detector 450 can calculate an average movement of the 3D points between frames. When a new frame is captured by the image capture devices 104, the movement detector 450 can overwrite (e.g., in memory) the 3D point cloud representing the previous frame with the current frame, and overwrite the current frame with the 3D point cloud of the new frame, and calculate the movement of the patient between the current and previous frames. The movement of the patient within the surgical environment over time can be stored in one or more data structures in the memory of the robot control system 405, such that the movement values (e.g., change in position, absolute position of the patient over time, etc.) are accessible to the components of the robot control system 405. In some implementations, the movement detector 450 can detect movement of the patient responsive to detecting that the movement exceeds a predetermined threshold (e.g., more than a few millimeters, etc.) from a previous frame or from a predetermined starting position (e.g., the position of the patient at the start of the procedure, etc.). In some implementations, the predetermined threshold can be defined as part of the target pathway or a surgical procedure associated with a target location in the surgical environment. For example, a surgeon may provide user input to define one or more thresholds as small if the robot is navigating in a region that requires high-precision movements. As such, the robot navigator 455 can maintain a data structure having a plurality of predetermined thresholds with respect to movement, each predetermined threshold assigned to a particular portion of a plurality of portions of image data corresponding to the patient, enabling the robot navigator 455 to control navigation in a manner that is more dynamic and responsive to underlying anatomical and surgical considerations.

The robot navigator 455 can generate, responsive to detecting the change in the position of the patient, instructions to modify the position of the surgical robot based on the change in position of the one or more points. As described herein above, the robot 420 can change its position by executing or interpreting instructions or signals indicating a target location for the instrument 430. For example, the robot 420 can execute such instructions that indicate a target location for the instrument 430, and actuate various movable components in the robot 420 to move the instrument 430 to the target location. The robot navigator 455 can navigate the robot 420 according to the movement of the patient such that the robot 420 or the instrument 430 is aligned with a target location or target pathway in the surgical environment, even when the patient is unsecured. The robot navigator 455 can use computer instructions to generate commands that cause movements of the robot 420 until, for example, a predetermined process or procedure is completed (e.g., the robot 420 or the instrument 430 has been navigated to one or more predetermined targets in the surgical environment) or an exit condition is triggered (e.g., a collaborative mode condition, etc.). In some implementations, the target location or the target pathway can be specified as part of the 3D image (e.g., the CT scan or the MRI scan, etc.) that is registered to the real-time 3D point cloud representing the patient in the frame of reference of the image capture devices 104. As the location of the instrument 430 and the robot 420 are also mapped within the same frame of reference, an accurate measure of the distance between the instrument 430 and the robot 420 can be determined. An offset may be added to this distance based on known attributes of the instrument, for example, to approximate the location of a predetermined portion of the instrument 430 (e.g., the tip or tool-end) within the surgical environment.

When movement of the patient is detected by the movement detector 450, the robot navigator 455 can generate corresponding instructions to move the robot 420 according to the patient movement. For example, from the CT scan or from user input, the instrument 430 may be aligned with a target pathway or target location in the surgical environment to carry out the surgical procedure. If the patient moves during the surgical procedure, the robot navigator 455 can generate instructions, for example, using one or more application programming interfaces (APIs) of the robot 430 to move the instrument 420 in-step with the detected patient movement. For example, if patient movement of 2 cm to the left of a target pathway is detected, the robot navigator 455 can adjust the position of the robot 430 the same 2 cm to the left, according to the target pathway. In some implementations, the robot navigator 455 can make adjustments while navigating the robot 420 along a predetermined trajectory (e.g., a pathway into the skull of the patient to reach a target location in the brain, etc.). For example, the robot 420 may be navigated left or right according to patient movement while also navigating the instrument 430 downward along a predetermined trajectory into the patient's skull. This provides an improvement over other robotic implementations that do not track and compensate for patient movement, as any patient movement during a procedure could result in patient harm. By moving the instrument in accordance with patient movement, unintended collisions or interference with other parts of the patient are mitigated, as the predetermined target pathway may be followed more exactly.

The robot navigator 455 can perform these adjustments iteratively, or periodically and multiple times per second to compensate for sudden patient movement. As described herein above, the movement detector 450 can calculate patient movement periodically (e.g., according to the capture rate of the image capture devices 104, etc.). Each time a new frame is captured by the image capture devices 104, the movement detector 450 can determine whether the change in the position of the patient satisfies a threshold (e.g., a predetermined amount of movement relative to a previous frame, to a patient position at the start of the procedure, or to the position of the instrument 430, etc.). The robot navigator 455 can then adjust the position of the robot 420 such that the instrument 430 is aligned with the predetermined pathway (e.g., a predetermined trajectory) relative to the detected change in the position of the patient. The change in the position of the trajectory or pathway can be determined based on a change in position or orientation of one or more target indicators in the 3D images (e.g., the CT scan or MRI scan, etc.) that are registered to the patient in real-time.

In some implementations, the 3D images can be modified to indicate the pathway or trajectory to the selected targets. Likewise, in implementations where multiple targets or pathways (or segments of pathways) are present, the surgeon may select one or more pathways along which the robot 420 should navigate the instrument 430 via user input (e.g., button selections, selections by touch screen, etc.). The robot navigator 455 can navigate the robot 420 along the selected pathways while compensating for patient movement in real-time, as described herein. The robot navigator 455 can navigate the robot 420 in a number of scenarios. For example, the robot navigator 455 can adjust the position of the robot 420 while the robot is still in space (e.g., providing a rigid port to the patient). In some implementations, the robot navigator 455 can adjust the position of the robot along one or more axes while an operator of the robot 420 is moving the robot 420 or the instrument 430 down to a target location through a predetermined trajectory.

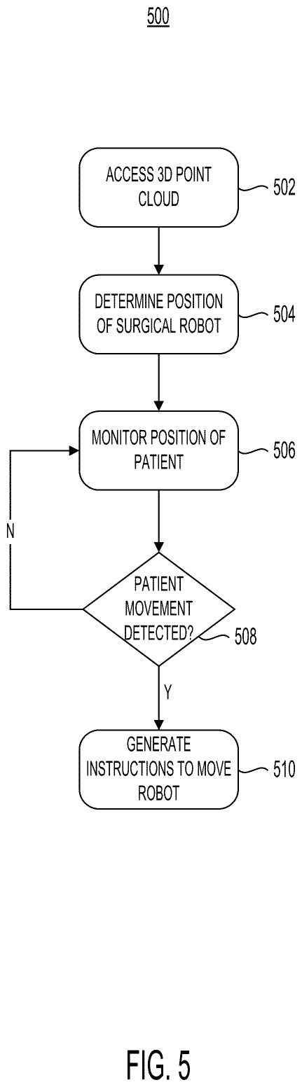

Referring now to FIG. 5, depicted is an example method 500 of controlling a surgical robot based on patient tracking techniques, in accordance with one or more implementations. The method 500 can be performed, for example, by the robot control systems 405, 605, or 805, or any other computing device described herein, including the computing system 1000 described herein in connection with FIGS. 12A and 12B. In brief overview of the method 500, at STEP 502, the robot control system (e.g., the robot control system 405, etc.) can access a 3D point cloud corresponding to a patient. At STEP 504, the robot control system can determine a position of a surgical robot (e.g., the robot 420). At STEP 506, the robot control system can monitor the position of a patient over time. At STEP 508, the robot control system can determine whether patient movement is detected. At STEP 510, the robot control system can generate instructions to move the robot according to the detected movement.

In further detail of the method 500, at STEP 502, the robot control system (e.g., the robot control system 405, etc.) can access a 3D point cloud corresponding to a patient. As described herein above, an image processing system (e.g. the image processing system 100) can utilize one or more image capture devices (e.g., the image capture devices 104), which can be 3D cameras, to capture a real-time (or near real-time) 3D image of a patient during a surgical procedure. The 3D point cloud can correspond, for example, to the head of the patient, the body of the patient, or to any other portion of the patient on which surgery can be performed. In some implementations, the image capture devices can be positioned in the surgical environment to capture an image of the patient's face. In some implementations, the robot control system can apply an image segmentation model to the 3D point cloud captured by the image capture devices. In some implementations, the robot control system can receive the point cloud from the processing circuitry of the image processing system, for example, via one or more communication interfaces. In some implementations, the robot control system can capture an indication of a global environment (e.g., a static point in the surgical environment from which the 3D point cloud corresponding to the patient may be accessed, etc.). The robot control system can receive 3D images including point clouds representing the patient iteratively, for example, at a predetermined framerate of the image capture devices. The robot control system can access or otherwise retrieve the 3D point clouds representing the user and store the 3D point clouds in the memory of the robot control system, such that they can be accessed by the other components of the robot control system. The 3D point clouds can be stored in association with a respective timestamp, such that the position and orientation of the patient can be determined in real-time or near real-time.

At STEP 504, the robot control system can determine a position of a surgical robot (e.g., the robot 420). In addition to tracking the position of the patient, the robot control system can determine a position of a surgical robot (e.g., the robot 420) within the same frame of reference as the 3D point cloud. As described herein above, the image capture devices can capture images of a patient in a surgical environment. The image capture devices can have a predetermined pose within the surgical environment relative to other sensors, such as IR sensors (e.g., the IR sensors 220). As described herein above, the IR sensors can be IR cameras that capture IR wavelengths of light. The robot control system can determine the position of the robot by utilizing one or more tracking techniques, such as IR tracking techniques, to determine the position of the robot. The robot can include one or more markers or indicators on its surface, such as IR indicators. The robot control system can utilize the IR sensors to determine the relative position of the robot. In some implementations, the robot control system can determine the orientation (e.g., the pose) of the robot by performing similar techniques. Because the sensors used to track the position of the robot are a known distance from the image capture devices, the position of the markers detected by the IR sensors can be mapped to the same frame of reference as the 3D point cloud captured by the image capture devices.

In some implementations, the image capture devices may be used to determine the position of the robot. For example, the robot can include one or more graphical indicators, such as bright distinct colors or QR codes, among others. In addition to capturing 3D point clouds of the patient, the image capture devices can capture images of the surgical environment, and the robot control system can perform image analysis techniques to determine the position of the surgical robot in the images based on the detected positions of the indicators in the images. The position and orientation of the surgical robot can be computed periodically, for example, in real-time or near real-time, enabling the robot control system to track the movement of the robot over time. In some implementations, the robot control system can perform a calibration procedure, for example, to establish the unified frame of reference between the 3D point clouds and the indicators coupled to the surgical robot. The calibration procedure can include identifying the position of the robot with respect to global indicators positioned in the surgical environment. The robot control system can store the position of the robot in the memory of the robot control system such that the real-time or near real-time position of the robot can be accessed by other components of the robot control system.

Tracking the position of the robot can include tracking the position of an instrument (e.g., the instrument 430) coupled to the robot. As described herein above, the instrument can include its own indicators that are coupled to the instrument or a bracket/connector coupling the instrument to the robot. An example of indicators coupled to the instrument is shown as the tracked instrument 340 in the close-up view 312 depicted in FIG. 3. The robot control system can track the position and orientation of the instrument as well as the robot in real-time or near real-time using techniques similar to those described above. The position and orientation of the instrument determined by the robot control system can be stored in the memory of the robot control system. In some implementations, the robot control system can store the position and orientation of the instrument in association with a respective timestamp.

At STEP 506, the robot control system can monitor the position of a patient over time. The robot control system can accurately detect patient movement and generate signals to adjust the position of the robot 420 to accommodate for patient movements. The robot control system can compare previous positions of the 3D point cloud representing the patient to the positions of current or new 3D point clouds captured by the image capture devices 104. In some implementations, the robot control system can maintain two sets of 3D point clouds: one from a previously captured frame and another from a currently captured (e.g., most-recently captured) frame. The comparison can be a distance (e.g., a Euclidean distance, etc.) in the frame of reference of the 3D point clouds. The robot control system can perform iterative computations to determine which 3D points in a first 3D point cloud (e.g., the previous frame) correspond to 3D points in a second 3D point cloud (e.g., the current frame). For example, the robot control system can perform an iterative ICP algorithm or a RANSAC fitting technique to approximate which points in the current frame correspond to the points in the previous frame. Distances between the corresponding points can then be determined. In some implementations, to improve computational performance, the robot control system can compare a subset of the points (e.g., by performing a down-sampling technique on the point clouds, etc.). For example, after finding the point correspondences, the robot control system can select a subset of the matching point pairs between each point cloud to compare (e.g., determine a distance between in space, etc.). In some implementations, to determine the movement of the patient in the surgical environment, the robot control system can calculate an average movement of the 3D points between frames.

At STEP 508, the robot control system can determine whether patient movement is detected. When a new frame is captured by the image capture devices 104, the robot control system can overwrite (e.g., in memory) the 3D point cloud representing the previous frame with the current frame, and overwrite the current frame with the 3D point cloud of the new frame, and calculate the movement of the patient between the current and previous frames. The movement of the patient within the surgical environment over time can be stored in one or more data structures in the memory of the robot control system, such that the movement values (e.g., change in position, absolute position of the patient over time, etc.) are accessible to the components of the robot control system. In some implementations, the robot control system can detect movement of the patient if the movement exceeds a predetermined threshold (e.g., more than a few millimeters, etc.) from a previous frame or from a predetermined starting position (e.g., the position of the patient at the start of the procedure, etc.). If the threshold is exceeded, the robot control system can generate instructions to move the robot at STEP 510. If the threshold is not exceeded, the robot control system can continue to monitor the patient movement at STEP 506.

At STEP 510, the robot control system can generate instructions to move the robot according to the detected movement. As described herein above, the robot can change its position by executing or interpreting instructions or signals indicating a target location for the instrument. For example, the robot can execute such instructions that indicate a target location for the instrument, and actuate various movable components in the robot to move the instrument to the target location. The robot control system can navigate the robot according to the movement of the patient such that the robot or the instrument is aligned with a target location or target pathway in the surgical environment, even when the patient is unsecured. In some implementations, the target location or the target pathway can be specified as part of the 3D image (e.g., the CT scan or the MRI scan, etc.) that is registered to the real-time 3D point cloud representing the patient in the frame of reference of the image capture devices. As the location of the instrument and the robot are also mapped within the same frame of reference, an accurate measure of the distance between the instrument and the can be determined. An offset may be added to this distance based on known attributes of the instrument, for example, to approximate the location of a predetermined portion of the instrument (e.g., the tip or tool-end) within the surgical environment.

When movement of the patient is detected by the robot control system, the robot control system can generate corresponding instructions to move the robot according to the patient movement. For example, from the CT scan or from user input, the instrument may be aligned with a target pathway or target location in the surgical environment to carry out the surgical procedure. If the patient moves during the surgical procedure, the robot control system can generate instructions, for example, using one or more application programming interfaces (APIs) of the robot to move the instrument in-step with the detected patient movement. For example, if patient movement of 2 cm to the left of a target pathway is detected, the robot control system can adjust the position of the robot the same 2 cm to the left, according to the target pathway. In some implementations, the robot control system can make adjustments while navigating the robot along a predetermined trajectory (e.g., a pathway into the skull of the patient to reach a target location in the brain, etc.). For example, the robot may be navigated left or right according to patient movement while also navigating the instrument downward along a predetermined trajectory into the patient's skull. This provides an improvement over other robotic implementations that do not track and compensate for patient movement, as any patient movement during a procedure could result in patient harm. By moving the instrument in accordance with patient movement, unintended collisions or interference with other parts of the patient are mitigated, as the predetermined target pathway may be followed more exactly.

The robot control system can perform these adjustments iteratively, or periodically and multiple times per second to compensate for sudden patient movement. As described herein above, the robot control system can calculate patient movement periodically (e.g., according to the capture rate of the image capture devices, etc.). Each time a new frame is captured by the image capture devices, the robot control system can determine whether the change in the position of the patient satisfies a threshold (e.g., a predetermined amount of movement relative to a previous frame, to a patient position at the start of the procedure, or to the position of the instrument, etc.). The robot control system can then adjust the position of the robot such that the instrument is aligned with the predetermined pathway (e.g., a predetermined trajectory) relative to the detected change in the position of the patient. The change in the position of the trajectory or pathway can be determined based on a change in position or orientation of one or more target indicators in the 3D images (e.g., the CT scan or MRI scan, etc.) that are registered to the patient in real-time. In some implementations, the 3D images can be modified to indicate the pathway or trajectory to the selected targets. Likewise, in implementations where multiple targets or pathways (or segments of pathways) are present, the surgeon may select one or more pathways along which the robot should navigate the instrument via user input (e.g., button selections, selections by touch screen, etc.). The robot control system can navigate the robot along the selected pathways while compensating for patient movement in real-time, as described herein.

### C. Controlling a Surgical Robot Based On Torque Sensing Techniques

The systems and methods described herein provide various techniques for controlling a surgical robot in a surgical environment. This section describes such techniques that operate in connection with torque sensors, which may be positioned, for example, on a patient or a surgical robot operating on a patient. The techniques described herein provide improved movement tracking and adjustment for surgical robots based on real-time torque measurements of forces applied by a patient. Torque measurements detected by the systems and methods described herein can be used to modify the position or orientation of a surgical tool during a surgical operation, which provides improvements to patient safety during surgical procedures. The torque sensors described herein may be positioned on various locations on the body of the patient or around the surgical site of the patient. In some implementations, other types of sensors, such as accelerometers, magnetometers, gyroscopes, or all-in-one sensors such as IMUs can be utilized in connection with or in place of the torque sensors.

Referring now to FIG. 6, depicted is an example system 600 for controlling a surgical robot (e.g., the robot 310, the robot 420, etc.) based on torque sensing techniques, in accordance with one or more implementations. The system 600 can include at least one robot control system 605, at least one robot 620, at least one image processing system 100, and one or more sensors 655. The robot control system 605 can include at least one measurement identifier 635, at least one robot tracker 640, at least one movement detector 645, and at least one robot navigator 650. The robot 620 can include an instrument 630.

Each of the components (e.g., the robot control system 605, the image processing system 100, the robot 620, etc.) of the system 600 can be implemented using the hardware components or a combination of software with the hardware components of a computing system (e.g., computing system 1000, any other computing system described herein, etc.) detailed herein in conjunction with FIGS. 12A and 12B. Each of the components of the robot control system 605 (e.g., the measurement identifier 635, the robot tracker 640, the movement detector 645, the robot navigator 650, etc.) can perform the functionalities detailed herein. It should be understood that although the imaging processing system 100 and the robot control system 605 are depicted as separate systems, that the robot control system 605 may be a part of the image processing system 100 (e.g., implemented at least in part by the processing circuitry 212, etc.) or vice versa (e.g., the processing circuitry 212 of the image processing system 100 implemented on one or more processors of the robot control system 605). Similarly, the robot control system 605 may be implemented with or may include the image processing system 1000 described in connection with FIG. 10, or vice versa. In implementations where the image processing system 100 and the robot control system 605 are implemented as separate computing systems, the image processing system 100 and the robot control system 605 can exchange information via a communications interface, as described herein. Likewise, the robot control system 605 and the robot 620 can communicate via one or more communications interfaces. The robot control system 605 can communicate any generated instructions to the robot 620 for execution.

The robot 620 and the instrument 630 can be similar to, and include any of the structure and functionality of, the robot 420 and the instrument 430, respectively. In addition, the robot control system 605 can include any of the structure or functionality of the robot control system 605. The robot control system 605 can be, or form a part of, the image processing system 100 described herein in conjunction with FIGS. 1A, 1B, and 2, and can perform any of the functionalities of the image processing system 100 as described herein. The robot control system 605 can include at least one processor and a memory (e.g., a processing circuit). The memory can store processor-executable instructions that, when executed by processor, cause the processor to perform one or more of the operations described herein. The processor can include a microprocessor, an ASIC, an FPGA, a GPU, etc., or combinations thereof. The memory can include, but is not limited to, electronic, optical, magnetic, or any other storage or transmission device capable of providing the processor with program instructions. The memory can further include a floppy disk, CD-ROM, DVD, magnetic disk, memory chip, ASIC, FPGA, ROM, RAM, EEPROM, EPROM, flash memory, optical media, or any other suitable memory from which the processor can read instructions. The instructions can include code from any suitable computer programming language. The robot control system 605 can include one or more computing devices or servers that can perform various functions as described herein. The robot control system 605 can include any or all of the components and perform any or all of the functions of the computer system 1000 described herein in connection with FIGS. 12A and 12B.

The sensors 655 can be any type of sensors that can detect movement from a patient, for example, in a surgical environment. The sensors 655 can be force transducers that convert detected mechanical force, such as torque, to an analog or digital signal. This signal can be communicated to the robot control system 605 via one or more communications interfaces. In general, the sensors 655 can monitor conditions of the surgical environment continuously (e.g., in real-time or near real-time, etc.), or at a predetermined periodic interval. In some implementations, the sensors 655 may provide a signal to the robot control system 605 in response to detecting torque or movement that exceeds a predetermined threshold. In addition to measuring torque, the sensors 655 can include any type of movement sensors, such as torque sensors, accelerometers, gyroscopes, magnetometers, or all-in-one sensors such as IMUs. Measurements from the sensors can be communicated to the robot control system 605 in response to a request, at predetermined time periods, or in response to detecting a signal that exceeds a predetermined threshold. In some implementations, one or more of the sensors 655 can be positioned one or more portions of the robot 620 or the instrument 630.

Referring now to the operations of the robot control system 605, the measurement identifier 635 can identify measurements captured by the sensors 655, and store the sensor measurements in the memory of the robot control system 605. In some implementations, the measurement identifier 635 can periodically transmit requests to the sensors 655, for example, at predetermined time periods. In response to the requests, the sensors 655 can transmit the measurement values for each sensor 655 to the measurement identifier 635. The measurement identifier 635 can store the sensor measurements, for example, chronologically, or otherwise in association with a timestamp corresponding to when the sensor measurement was captured. In some implementations, the measurement identifier 635 can store each sensor measurement in association with an identifier of the sensor 655 from which the measurement was captured. In some implementations, measurement identifier 635 can store an association between each measurement and the object (e.g., the patient, the robot 620, the instrument 630, etc.) to which the respective sensor 655 is coupled. This allows the components of the robot control system 605 to measure or detect forces experienced or produced by each object in the surgical environment.

The robot tracker 640 can determine a position of the robot 620 within the surgical environment. As described herein above, the image capture devices 104 can capture images of a patient in a surgical environment. The image capture devices 104 can have a predetermined pose within the surgical environment relative to other sensors, such as the IR sensors 220. As described herein above, the IR sensors 220 can be IR cameras that capture IR wavelengths of light. The robot tracker 640 can determine the position of the robot by utilizing one or more tracking techniques, such as IR tracking techniques or computer vision tracking techniques, to determine the position of the robot 620. The robot 620 can include one or more markers or indicators on its surface, such as IR indicators. The robot tracker 640 can utilize the IR sensors 220 to determine the relative position of the robot 620 in the surgical environment. In some implementations, the robot tracker 640 can determine the orientation (e.g., the pose) of the robot 620 by performing similar techniques. Because the IR sensors 220 (or other sensors) used to track the position of the robot 620 are a known distance from the image capture devices 104, the position of the markers detected by the IR sensors 220 can be mapped to the same frame of reference as the 3D point cloud captured by the image capture devices 104.

In some implementations, the image capture devices 104 may be used to determine the position of the robot 620. For example, the robot 620 can include one or more graphical indicators, such as bright distinct colors, patterns, or QR codes, among others. The image capture devices 104 can capture images of the surgical environment, and the robot tracker 640 can perform image analysis techniques to determine the position of the surgical robot in the images 208 based on the detected positions of the indicators in the images 208. The position and orientation of the surgical robot can be computed periodically, for example, in real-time or near real-time, enabling the robot tracker 620 to track the movement of the robot 620 over time. In some implementations, the robot tracker 620 can perform a calibration procedure, for example, to establish the unified frame of reference between the 3D point clouds and the indicators coupled to the surgical robot. The calibration procedure can include identifying the position of the robot 620 with respect to global indicators positioned in the surgical environment. The robot tracker 640 can store the position of the robot 620 in the memory of the robot tracker 640 such that the real-time or near real-time position of the robot 620 can be accessed by other components of the robot control system 605. The calibration procedure can include using predetermined markers or patterns in the surgical environment (e.g., a chessboard pattern, etc.) to calibrate the pose (e.g., the position and orientation) of the robot 620 with respect to the patient.

Tracking the position of the robot 620 can include tracking the position of the instrument 630. As described herein above, the instrument 630 can include its own indicators that are coupled to the instrument 630 or a bracket/connector coupling the instrument 630 to the robot 620. An example of indicators coupled to the instrument 630 is shown as the tracked instrument 340 in the close-up view 312 depicted in FIG. 3. The robot tracker 640 can track the position and orientation of the instrument 630 as well as the robot 620 in real-time or near real-time using techniques similar to those described above. The position and orientation of the instrument 630 determined by the robot tracker 640 can be stored in the memory of the robot control system 605. In some implementations, the robot tracker 640 can store the position and orientation of the instrument 630 in association with a respective timestamp. In some implementations, tracking the position of the robot 620 or the instrument 630 can also be performed based on the measurements captured from one or more sensors 655 coupled to the robot 620 or the instrument 630. For example, the robot tracker 640 may determine the position of the robot 620 using, for example, acceleration or velocity values (captured from the sensors 655) to interpolate the position of the robot 620 or the instrument 630 over time.

The movement detector 645 can detect a position modification condition based on the set of measurements captured by the one or more sensors 655. As described herein above, in some implementations, one or more of the sensors 655 can be positioned on one or more portions of the patient. The measurements captured from these sensors 655 can indicate to the movement detector 645 that the patient's position has changed over time (e.g., experienced acceleration, exerted a force or torque since the start of the surgical procedure, etc.). The movement detector 645 can detect the movement of the patient by integrating (e.g., in the case of acceleration or velocity measurements) or interpolating (e.g., in the case of position measurements, etc.) the position of the patient over time. The movement detector 645 can store the detected position of the patient in association with a respective timestamp corresponding to when the corresponding sensor measurements were captured. In this way, the movement detector 645 can determine and record the position of the patient over time in the surgical environment.

The movement detector 645 can detect whether the position of the patient has satisfied a position modification condition. The position modification condition can be a predetermined amount of movement or deviation from initial conditions of a surgical procedure. Using the recorded position values of the patient determined from the measurements of the sensors 655, the movement detector 645 can determine an amount of movement over time by comparing the current (e.g., from the most-recent sensor measurements) position of the patient to initial conditions of the patient. If the position modification condition is satisfied, the robot navigator 650 can adjust the position of the robot 620 or the instrument 630 to minimize the occurrence of injury to the patient.

In some implementations, the movement detector 645 can determine that the position modification conditions has been satisfied if the determined movement of the patient over time is greater than or equal to a predetermined threshold amount of movement. In some implementations, the movement of the patient can be measured from position of the patient following the previous position adjustment of the robot 620, such that the adjusted position becomes the new baseline for subsequent adjustments of the position of the robot 620 or the instrument 630. As described herein above, the robot 620 can be navigated along a predetermined trajectory such that the instrument 630 can interact with a target location to carry out a portion of a surgical operation. In some implementations, the movement detector 645 can detect that the position modification condition has been satisfied by determining that a position of the surgical robot has deviated from a predetermined trajectory. For example, the measurements of one or more sensors 655 positioned on the robot 620 can indicate that the robot 620 has experienced an external force (e.g., from a collision with another object, etc.) that has caused or will cause the robot 620 to deviate from the predetermined trajectory. In response to the detected force, the movement detector 645 can generate a signal that causes the robot control system 605 to navigate the robot 620 back to the predetermined trajectory.

In some implementations, one or more of the measurements from the sensors 655 can indicate that a collision has occurred between the robot 620 or the instrument 630 and another object in the surgical environment. For example, one or more force sensors can indicate that a collision has occurred against a surface of the patient. In some implementations, the movement detector 645 can detect that the position modification condition has been satisfied by determining that a collision occurred with the robot 620 (or the instrument 630) based on the set of measurements. If a collision occurs, the movement detector 645 can generate a signal, which may include information relating to the nature of the collision (e.g., the direction of force experienced, a direction that the robot 620 should be moved to avoid further or harmful collisions, etc.). This information can be used by the robot control system 605 to navigate the robot 620 to avoid further collisions.

The robot navigator 650 can generate instructions to modify the position of the surgical robot based on the set of measurements. As described herein, once the position modification condition has been satisfied, the robot navigator 650 can generate instructions for the robot 620 corresponding to the sensor measurements that triggered the position modification condition. As described herein above, the robot 620 can change its position by executing or interpreting instructions or signals indicating a target location for the instrument 630. For example, the robot 620 can execute such instructions that indicate a target location for the instrument 630, and actuate various movable components in the robot 620 to move the instrument 630 to the target location. The robot navigator 650 can navigate the robot 620 according to the movement of the patient such that the robot 620 or the instrument 630 is aligned with a target location or target pathway in the surgical environment, even when the patient is unsecured. In some implementations, the target location or the target pathway can be specified as part of a 3D image (e.g., the CT scan or the MRI scan, etc.), as described herein above in connection with FIG. 4.

When movement of the patient is detected by the movement detector 645, the robot navigator 650 can generate corresponding instructions to move the robot 620 according to the patient movement. For example, the sensor measurements monitored by the robot tracker 640 can indicate that the patient has moved by a determined amount in a determined direction. The robot navigator 650 can generate instruments for the robot to move the instrument 630 in accordance with the movement of the patient, such that the instrument 630 remains on a predetermined pathway or trajectory that leads to a selected target location on or within the patient. In some implementations, the robot navigator 650 can generate instructions to compensate from an external force experienced by the robot 620. For example, the instructions can cause the robot 620 to move against the detected force, in order to remain in the proper position relative to the patient.

The robot navigator 650 can perform these adjustments iteratively, or periodically and multiple times per second to compensate for sudden patient movement or detected force. As described herein above, the movement detector 645 can calculate patient movement periodically (e.g., according to the rate at which the sensors 655 capture measurements, etc.). Each time new measurements from the sensors 655 are captured, the movement detector 645 can determine whether the change in the position of the patient (or the robot 620) satisfies the position modification condition. The robot navigator 650 can then adjust the position of the robot 620 such that the instrument 630 is aligned with the predetermined pathway (e.g., a predetermined trajectory) based on the conditions that triggered the position modification condition (e.g., the measurements from the sensors 655). In addition, the robot navigator 650 can navigate the robot 620 along target pathways while compensating for patient movement in real-time, as described herein. The robot navigator 650 can navigate the robot 620 in a number of scenarios. For example, the robot navigator 650 can adjust the position of the robot 620 while the robot 620 is still in space (e.g., providing a rigid port to the patient). In some implementations, the robot navigator 650 can adjust the position of the robot along one or more axes while an operator of the robot 620 is moving the robot 620 or the instrument 630 down to a target location through a predetermined trajectory.

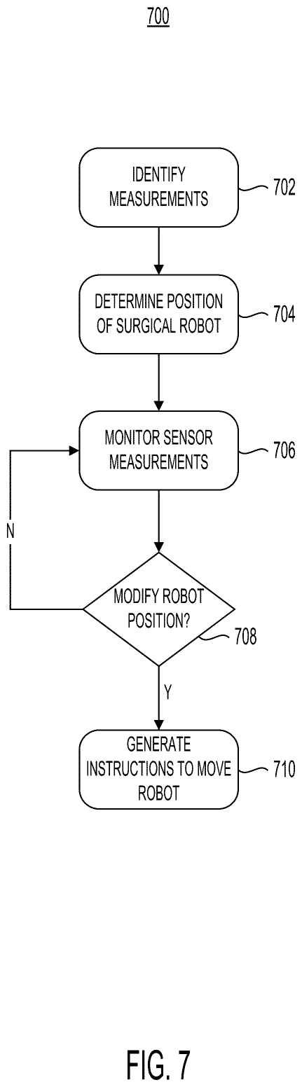

Referring now to FIG. 7, depicted is an example method 700 of controlling a surgical robot based on patient tracking techniques, in accordance with one or more implementations. The method 700 can be performed, for example, by the robot control systems 405, 605, or 805, or any other computing device described herein, including the computing system 1000 described herein in connection with FIGS. 12A and 12B. In brief overview of the method 700, at STEP 702, the robot control system (e.g., the robot control system 605, etc.) can identify measurements from sensors (e.g., the sensors 655, etc.). At STEP 704, the robot control system can determine a position of a surgical robot (e.g., the robot 620). At STEP 706, the robot control system can monitor the sensor measurements. At STEP 708, the robot control system can determine whether a position modification condition is satisfied. At STEP 710, the robot control system can generate instructions to move the robot.

In further detail of the method 700, at STEP 702, the robot control system (e.g., the robot control system 605, etc.) can identify measurements from sensors (e.g., the sensors 655, etc.). The robot control system can store the sensor measurements in the memory of the robot control system. In some implementations, the robot control system can periodically transmit requests to the sensors, for example, at predetermined time periods. In response to the requests, the sensors can transmit the measurement values for each sensor to the robot control system. The robot control system can store the sensor measurements, for example, chronologically, or otherwise in association with a timestamp corresponding to when the sensor measurement was captured. In some implementations, the robot control system can store each sensor measurement in association with an identifier of the sensor from which the measurement was captured. In some implementations, robot control system can store an association between each measurement and the object (e.g., the patient, the robot, an instrument such as the instrument 630, etc.) to which the respective sensor is coupled. This allows the components of the robot control system to measure or detect forces experienced or produced by each object in the surgical environment.

At STEP 704, the robot control system can determine a position of a surgical robot (e.g., the robot 620). As described herein above, image capture devices (e.g., the image capture devices 104) can capture images of a patient in a surgical environment. The image capture devices can have a predetermined pose within the surgical environment relative to other sensors, such as IR sensors (e.g., the IR sensors 220). As described herein above, the IR sensors can be IR cameras that capture IR wavelengths of light. The robot control system can determine the position of the robot by utilizing one or more tracking techniques, such as IR tracking techniques or computer vision tracking techniques, to determine the position of the robot. The robot can include one or more markers or indicators on its surface, such as IR indicators. The robot control system can utilize the IR sensors to determine the relative position of the robot in the surgical environment. In some implementations, the robot control system can determine the orientation (e.g., the pose) of the robot by performing similar techniques. Because the IR sensors (or other sensors) used to track the position of the robot are known distances from the image capture devices, the position of the markers detected by the IR sensors can be mapped to the same frame of reference as the 3D point cloud captured by the image capture devices.

In some implementations, the image capture devices may be used to determine the position of the robot. For example, the robot can include one or more graphical indicators, such as bright distinct colors, patterns, or QR codes, among others. The image capture devices can capture images of the surgical environment, and the robot control system can perform image analysis techniques to determine the position of the surgical robot in the images 208 based on the detected positions of the indicators in the images. The position and orientation of the surgical robot can be computed periodically, for example, in real-time or near real-time, enabling the robot control system to track the movement of the robot over time. In some implementations, the robot control system can perform a calibration procedure, for example, to establish the unified frame of reference between the 3D point clouds and the indicators coupled to the surgical robot. The calibration procedure can include identifying the position of the robot with respect to global indicators positioned in the surgical environment. The robot control system can store the position of the robot in the memory of the robot control system such that the real-time or near real-time position of the robot can be accessed by other components of the robot control system. The calibration procedure can include using predetermined markers or patterns in the surgical environment (e.g., a chessboard pattern, etc.) to calibrate the pose (e.g., the position and orientation) of the robot with respect to the patient.

Tracking the position of the robot can include tracking the position of an instrument (e.g. the instrument 630). As described herein above, the instrument can include its own indicators that are coupled to the instrument or a bracket/connector coupling the instrument to the robot. An example of indicators coupled to the instrument is shown as the tracked instrument 340 in the close-up view 312 depicted in FIG. 3. The robot control system can track the position and orientation of the instrument as well as the robot in real-time or near real-time using techniques similar to those described above. The position and orientation of the instrument determined by the robot control system can be stored in the memory of the robot control system. In some implementations, the robot control system can store the position and orientation of the instrument in association with a respective timestamp. In some implementations, tracking the position of the robot or the instrument can also be performed based on the measurements captured from one or more sensors coupled to the robot or the instrument. For example, the robot control system may determine the position of the robot using, for example, acceleration or velocity values (captured from the sensors) to interpolate the position of the robot or the instrument over time.

At STEP 706, the robot control system can monitor the sensor measurements. As described herein above, in some implementations, one or more of the sensors can be positioned on one or more portions of the patient. The measurements captured from these sensors can indicate to the robot control system that the patient's positioned has changed over time (e.g., experienced acceleration, exerted a force or torque since the start of the surgical procedure, etc.). The robot control system can detect the movement of the patient by integrating (e.g., in the case of acceleration or velocity measurements) or interpolating (e.g., in the case of position measurements, etc.) the position of the patient over time. The robot control system can store the detected position of the patient in association with a respective timestamp corresponding to when the corresponding sensor measurements were captured. In this way, the robot control system can determine and record the position of the patient over time in the surgical environment.

At STEP 708, the robot control system can determine whether a position modification condition is satisfied. The robot control system can detect whether the position of the patient has satisfied a position modification condition. The position modification condition can be a predetermined amount of movement or deviation from initial conditions of a surgical procedure. Using the recorded position values of the patient determined from the measurements of the sensors, the robot control system can determine an amount of movement over time by comparing the current (e.g., from the most-recent sensor measurements) position of the patient to initial conditions of the patient. If the position modification condition is satisfied, the robot navigator can adjust the position of the robot or the instrument to minimize the occurrence of injury to the patient.

In some implementations, the robot control system can determine that the position modification conditions has been satisfied if the determined movement of the patient over time is greater than or equal to a predetermined threshold amount of movement. In some implementations, the movement of the patient can be measured from position of the patient following the previous position adjustment of the robot, such that the adjusted position becomes the new baseline for subsequent adjustments of the position of the robot or the instrument. As described herein above, the robot can be navigated along a predetermined trajectory such that the instrument can interact with a target location to carry out a portion of a surgical operation. In some implementations, the robot control system can detect that the position modification condition has been satisfied by determining that a position of the surgical robot has deviated from a predetermined trajectory. For example, the measurements of one or more sensors positioned on the robot can indicate that the robot has experienced an external force (e.g., from a collision with another object, etc.) that has caused or will cause the robot to deviate from the predetermined trajectory. In response to the detected force, the robot control system can generate a signal that causes the robot control system to navigate the robot back to the predetermined trajectory.

In some implementations, one or more of the measurements from the sensors can indicate that a collision has occurred between the robot or the instrument and another object in the surgical environment. For example, one or more force sensors can indicate that a collision has occurred against a surface of the patient. In some implementations, the robot control system can detect that the position modification condition has been satisfied by determining that a collision occurred with the robot (or the instrument) based on the set of measurements. If a collision occurs, the robot control system can generate a signal, which may include information relating to the nature of the collision (e.g., the direction of force experienced, a direction that the robot should be moved to avoid further or harmful collisions, etc.). This information can be used by the robot control system to navigate the robot to avoid further collisions. If the position modification condition has been satisfied, the robot control system can execute STEP 710 of the method 700. If the position modification condition has not been satisfied, the robot control system can continue to monitor the sensor measurements at STEP 706 of the method 700.

At STEP 710, the robot control system can generate instructions to move the robot. As described herein, once the position modification condition has been satisfied, the robot control system can generate instructions for the robot corresponding to the sensor measurements that triggered the position modification condition. As described herein above, the robot can change its position by executing or interpreting instructions or signals indicating a target location for the instrument. For example, the robot can execute such instructions that indicate a target location for the instrument, and actuate various movable components in the robot to move the instrument to the target location. The robot control system can navigate the robot according to the movement of the patient such that the robot or the instrument is aligned with a target location or target pathway in the surgical environment, even when the patient is unsecured.

When movement of the patient is detected by the robot control system, the robot control system can generate corresponding instructions to move the robot according to the patient movement. For example, the sensor measurements monitored by the robot tracker 640 can indicate that the patient has moved by a determined amount in a determined direction. The robot control system can generate instruments for the robot to move the instrument in accordance with the movement of the patient, such that the instrument remains on a predetermined pathway or trajectory that leads to a selected target location on or within the patient. In some implementations, the robot control system can generate instructions to compensate from an external force experienced by the robot. For example, the instructions can cause the robot to move against the detected force, in order to remain in the proper position relative to the patient.

The robot control system can perform these adjustments iteratively, or periodically and multiple times per second to compensate for sudden patient movement or detected force. As described herein above, the robot control system can calculate patient movement periodically (e.g., according to the rate at which the sensors capture measurements, etc.). Each time new measurements from the sensors are captured, the robot control system can determine whether the change in the position of the patient (or the robot) satisfies the position modification condition. The robot control system can then adjust the position of the robot such that the instrument is aligned with the predetermined pathway (e.g., a predetermined trajectory) based on the conditions that triggered the position modification condition (e.g., the measurements from the sensors). In addition, the robot control system can navigate the robot along target pathways while compensating for patient movement in real-time, as described herein. The robot control system can navigate the robot in a number of scenarios. For example, the robot control system can adjust the position of the robot while the robot is still in space (e.g., providing a rigid port to the patient). In some implementations, the robot control system can adjust the position of the robot along one or more axes while an operator of the robot is moving the robot or the instrument down to a target location through a predetermined trajectory.

### D. Initiating Collaborative Control in Response to Detected Conditions

The robotic systems described herein may operate autonomously, or may operate in connection with input from a surgeon. For example, the surgical robots described herein may aid guiding a surgical instrument along a predetermined pathway, while the surgeon can manually insert or remove the instrument from the patient, as well as activate the instrument to carry out a surgical procedure. However, in some cases, it is desirable for a surgeon to have full manual control over the position and trajectory of a surgical instrument. The systems and methods described herein provide improved techniques for detecting and managing such conditions. The techniques described herein can be used to detect conditions where manual control over a surgical instrument should be established, and generate instructions for a surgical robot to initiate manual control. The manual control may be referred to as “collaborative control,” as the surgical robot may still support the weight of the instrument while the position of the instrument may be guided manually by a surgeon. These and other improvements are detailed herein below.

Referring now to FIG. 8, depicted is an example system 800 for initiating collaborative control of a surgical robot (e.g., the robot system 300 or components thereof, such as robotic arm 310; the system 400 or components thereof, such as the robot 420; the system 600 or components thereof, such as the robot 620, etc.) in response to detected conditions, in accordance with one or more implementations. The system 800 can include at least one robot control system 805, at least one robot 820, at least one image processing system 100, and one or more sensors 855. Similar to various robots described herein, the robot 820 can include one or more members that can be manipulated manually and/or automatically using various actuators in response to external forces and/or control signals. The robot control system 805 can include at least one robot navigator 835, at least one control condition detector 840, and at least one manual control initiator 845. The robot 820 can include an instrument 830.

Each of the components (e.g., the robot control system 805, the image processing system 100, the robot 820, etc.) of the system 800 can be implemented using the hardware components or a combination of software with the hardware components of a computing system (e.g., computing system 1200 described in connection with FIGS. 12A and 12B). Each of the components of the robot control system 805 (e.g., the robot navigator 835, the control condition detector 840, the manual control initiator 845, etc.) can perform the functionalities detailed herein. It should be understood that although the imaging processing system 100 and the robot control system 805 are depicted as separate systems, that the robot control system 805 may be a part of the image processing system 100 (e.g., implemented at least in part by the processing circuitry 212, etc.) or vice versa (e.g., the processing circuitry 212 of the image processing system 100 implemented on one or more processors of the robot control system 805). Similarly, the robot control system 805 may be implemented with or may include the image processing system 1000 described in connection with FIG. 10. In implementations where the image processing system 100 and the robot control system 805 are implemented as separate computing systems, the image processing system 100 and the robot control system 805 can exchange information via a communications interface, as described herein. Likewise, the robot control system 805 and the robot 820 can communicate via one or more communications interfaces. The robot control system 805 can communicate any generated instructions to the robot 820 for execution.

The robot 820 and the instrument 830 can be similar to, and include any of the structure and functionality of, any of the robots or instruments described herein (e.g., the robots 420 or 620, the instruments 430 or 630, etc.). The sensors 855 can be similar to, and include any of the structure and functionality of, the sensors 655 described herein above in connection with FIG. 6. In addition, the robot control system 805 can include any of the structure or functionality of the robot control systems 405 or 605. The robot control system 805 can be, or form a part of, the image processing system 100 described herein in conjunction with FIGS. 1A, 1B, and 2, and can perform any of the functionalities of the image processing system 100 as described herein. The robot control system 805 can include at least one processor and a memory (e.g., a processing circuit). The memory can store processor-executable instructions that, when executed by processor, cause the processor to perform one or more of the operations described herein. The processor can include a microprocessor, an ASIC, an FPGA, a GPU, etc., or combinations thereof. The memory can include, but is not limited to, electronic, optical, magnetic, or any other storage or transmission device capable of providing the processor with program instructions. The memory can further include a floppy disk, CD-ROM, DVD, magnetic disk, memory chip, ASIC, FPGA, ROM, RAM, EEPROM, EPROM, flash memory, optical media, or any other suitable memory from which the processor can read instructions. The instructions can include code from any suitable computer programming language. The robot control system 805 can include one or more computing devices or servers that can perform various functions as described herein. The robot control system 805 can include any or all of the components and perform any or all of the functions of the computer system 1000 described herein in connection with FIGS. 12A and 12B.

Referring now to the operations of the robot control system 805, the robot navigator 835 can control a position of the robot 820 and the instrument 830 in a surgical environment including a patient. For example, the robot navigator 835 can identify one or more predetermined trajectories along which the instrument 830 should be guided in order to carry out a surgical procedure. In some implementations, the surgeon pre-plans the one or more trajectories, and can map the trajectories to one or more points of interest in a 3D image of the patient (e.g., a CT scan or an MRI scan), as described herein. When conducting the procedure, in implementations where multiple targets or pathways (or segments of pathways) are present, the surgeon may select one or more pathways along which the robot 820 should navigate the instrument 830 via user input (e.g., button selections, selections by touch screen, etc.). The robot navigator 835 can receive the user input and identify the one or more pathways by processing the user input. The robot navigator 835 can navigate the robot 820 along the selected pathways while compensating for patient movement in real-time, as described herein.

The robot navigator 835 can navigate the robot 820 in a number of scenarios. For example, the robot navigator 835 can adjust the position of the robot 820 while the robot is still in space (e.g., providing a rigid port to the patient). In some implementations, the robot navigator 835 can adjust the position of the robot along one or more axes while an operator of the robot 820 is moving the robot 820 or the instrument 830 down to a target location through a predetermined trajectory. The robot navigator 835 can further navigate the robot 820 using the techniques described herein above in connection with FIGS. 4 and 6. For example, the robot navigator 835 can generate instructions to adjust the position of the robot 820 in response to a 3D point cloud representing the patient indicates that the patient has moved. In addition, the robot navigator 835 can generate instructions to adjust the position of the robot in response to conditions of measured signals from the sensors 855. In some implementations, the robot navigator 835 can navigate the robot 820 continuously or in near real-time, to closely match the conditions of the surgical environment (e.g., sudden patient movement, instantaneous forces experienced, etc.).

The control condition detector 840 can detect a collaborative control condition of the robot 820 based on one or more conditions of the surgical environment. As described herein above, the robot 820 can operate autonomously, semi-autonomously, or in manually, where the surgeon has complete control over the position and orientation of the robot 820 and the instrument 830. Under certain conditions, it is advantageous to initiate collaborative control of the robot, such that the surgeon has total manual control over the position and orientation of the surgical instrument. The control condition detector 840 can monitor the conditions of the surgical environment using, for example, information from the image processing system 100 or the sensors 855 to detect whether a collaborative control condition has been satisfied.

In some implementations, the control condition detector 840 can access and monitor 3D point clouds corresponding the patient via the image processing system 100. As described in greater detail herein above in connection with FIG. 4, the robot control system 805 (which can include any of the functionality of the robot control system 405) can receive 3D images corresponding to the patient, and identify one or more points in the 3D point cloud over time. By monitoring the positions of the points in the 3D point clouds, the control condition detector 840 can determine a change in position of the patient over time. In some cases, it is advantageous to allow the surgeon to have full manual control of the robot 820 and the instrument 830 when patient movement is detected that exceeds one or more thresholds. For example, in some implementations, if the patient moves by a predetermined displacement over a predetermined amount of time (e.g., a relatively large distance in a relatively short amount of time), the control condition detector 840 can determine that the control condition is satisfied, and generate a signal for the robot control system 805 to initiate collaborative control of the robot 820.

In some implementations, the control condition detector 840 can identify measurements produced by the sensors 855. For example, to identify measurements from the sensors 855, the control condition detector 840 can perform operations such as those described herein above in connection with the measurement identifier 635 of FIG. 6. For example, in some implementations, one or more of the sensors 855 can be positioned on or coupled to the patient during the surgical procedure. The control condition detector 840 can identify measurements from the sensors 855, for example, to determine an amount of patient movement over time. Using the recorded position values of the patient determined from the measurements of the sensors 855, the control condition detector 840 can determine an amount of movement over time by comparing the current (e.g., from the most-recent sensor measurements) position of the patient to initial conditions of the patient. The control condition detector 840 can compare the amount of patient movement to a patient movement condition (e.g., a predetermined threshold of patient movement over time that will trigger collaborative control). The control condition detector 840 can continuously or periodically monitor the measurements from the sensors 855 such that patient movement can be monitored in real-time. The control condition detector 840 can compare patient movement to the threshold each time it is detected, and generate a signal for the robot control system 805 to initiate collaborative control of the robot 820 if the movement condition has been satisfied.

In some implementations, one or more of the sensors 855 can be positioned at a junction between the robot 820 and the patient. For example, in some implementations, the robot 820 may be secured to the patient (e.g., screwed into a patient's skull, etc.). If the control condition detector 840 detects measurements from the force sensors 855 on the robot that indicate patient movement (e.g., that exceeds a threshold, etc.), the control condition detector 840 can generate a signal for the robot control system 805 to initiate collaborative control of the robot 820. In some implementations, one or more of the sensors 855 can be positioned on the instrument 830, such as on a tip of a needle forming a part of the instrument 830. The control condition detector 840 can detect that measurements from the sensor(s) 855 positioned at the needle tip (e.g., IMU sensors, accelerometers, torque sensors, etc.) indicate that the needle tip has deviated from the predetermined pathway (e.g., the tip has bent or deformed due to external force, etc.). In response, the control condition detector 840 can generate a signal for the robot control system 805 to initiate collaborative control of the robot 820.

In some implementations, the control condition detector 840 can detect the collaborative control condition based on signals received from the sensors 855 positioned on the robot 820 or the instrument 830. For example, in some implementations, the sensors 855 can have forces applied to the robot 820 by a surgeon operating the robot 820. If the surgeon applies a force that exceeds a predetermined threshold in a predetermined amount of time (e.g., a jerking motion, etc.), this can indicate that the surgeon is attempting to take manual control of the robot 820. In response, the control condition detector 840 can generate a corresponding signal to initiate collaborative control of the robot 820. The control condition detector 840 can also detect collision events throughout the robot 820 from sensors coupled to the robot 820. For example, if the robot 820 is a robotic arm, such as the robotic arm 310, one or more of the sensors 855 can be positioned at the robotic arm and provide measurements of external forces (e.g., collisions) experienced by the robot 820. The control condition detector 840 can detect any collisions with the robot 820 based on the measurements from the sensors 855 (e.g., measurements exceeding a threshold that indicate unexpected movement, etc.) and generate a signal for the robot control system 805 to initiate collaborative control of the robot 820.

The control condition detector 840 can detect the collaborative control condition based on an error condition in an image-to-patient registration process, such as the image-to-patient process performed by the image registration component 445 described herein above in connection with FIG. 4. As described herein, the robot control system 805 can perform an image-to-patient registration technique to align a 3D image of the patient, which may include indicators of potential targets, and the 3D point cloud captured from the patient. A 3D image, such as a CT scan of the patient's head, can include both a 3D representation of the patient's face and an indication of the biopsy region within the patient's brain. By registering the 3D image of the patient with the real-time 3D point cloud of the patient in the surgical environment, the robot control system 805 can map the 3D image of the patient, and any target indicators, into the same frame of reference as both the 3D point cloud, the tracked position of the robot 820, and the tracked position of the instrument 430. Doing so allows the robot control system 805 to track the position of the instrument 830 with respect to both the patient and the target regions indicated in the 3D image of the patient. To register the 3D image to the 3D point cloud of the patient, the robot control system 805 can perform an iterative fitting process, such as a RANSAC algorithm and an iterative closest-point algorithm. The robot control system 805 can continuously register the 3D image to the 3D point cloud of the patient. If registration fails (e.g., the fitting algorithm fails to fit the 3D image to the 3D point cloud within a predetermined error threshold, etc.), the robot control system 805 can generate a signal indicating the failure. In response to detecting the failure signal from the robot control system 805, the control condition detector 840 can generate a signal for the robot control system 805 to initiate collaborative control of the robot 820 if the condition has been satisfied.

In addition, a surgeon may manually initiate collaborative control of the robot 820. As described herein, the robot 820 can include one or more buttons, or may be control through one or more user interfaces, such as a touch screen. In some implementations, a button positioned on the robot 820 (such as one of the buttons 335 described herein in connection with FIG. 3), or an actionable object (e.g., a graphical button, a hyperlink, or other user interface element, etc.) on a user interface 120 can generate a signal to the control condition detector 840 to initiate collaborative control. The control condition detector 840 can receive a signal indicating an interaction with a button corresponding to the collaborative control condition, and generate a signal for the robot control system 805 to initiate collaborative control of the robot 820.

Upon detecting the signal generated by the control condition detector 840, the manual control initiator 845 can generate instructions for the robot 820 to provide manual control to a surgeon operating the robot 820. In some implementations, the manual control initiator 845 can store an indication of the collaborative control event, and any conditions that caused the collaborative control event to occur, in one or more data structures in the memory of the robot control system 805. In some implementations, the manual control initiator 845 can store the collaborative control event in association with a timestamp indicating when the collaborative control event occurred. The manual control initiator 845 can communicate the instructions, which may be generated using one or more APIs corresponding to the robot 820 using a communications interface. In some implementations, the manual control initiator 845 can receive an indication to re-initiate automatic navigation of the robot 820 (e.g., via user input from the surgeon, pressing a button, interaction at a user interface 120, etc.), and the manual control initiator 845 can generate a signal for the robot navigator 835 to continue navigating the robot according to a target trajectory.

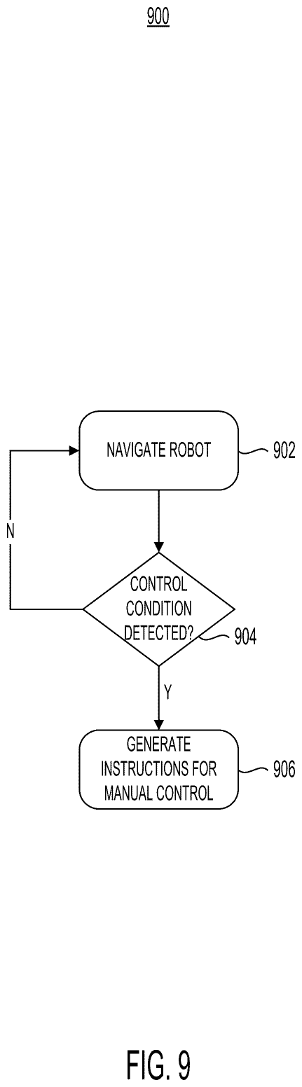

Referring now to FIG. 9, depicted is an example method 900 of controlling a surgical robot based on patient tracking techniques, in accordance with one or more implementations. The method 900 can be performed, for example, by the robot control systems 405, 605, or 805, or any other computing device described herein, including the computing system 1000 described herein in connection with FIGS. 12A and 12B. In brief overview of the method 900, at STEP 902, the robot control system (e.g., the robot control system 605, etc.) can navigate a surgical robot (e.g., the robot 820). At STEP 904, the robot control system can determine whether a collaborative control condition has been detected. At STEP 906, the robot control system can generate instructions to initiate collaborative control of the robot.

In further detail of the method 900, at STEP 902, the robot control system (e.g., the robot control system 605, etc.) can navigate a surgical robot (e.g., the robot 820). The robot control system can control a position of the robot and an instrument coupled to the robot (e.g., the instrument 830, etc.) in a surgical environment including a patient. For example, the robot control system can identify one or more predetermined trajectories along which the instrument should be guided in order to carry out a surgical procedure. In some implementations, the surgeon pre-plan the one or more trajectories, and can map the trajectories to one or more points of interest in a 3D image of the patient (e.g., a CT scan or an MRI scan), as described herein. When conducting the procedure, in implementations where multiple targets or pathways (or segments of pathways) are present, the surgeon may select one or more pathways along which the robot should navigate the instrument via user input (e.g., button selections, selections by touch screen, etc.). The robot control system can navigate the robot along the selected pathways while compensating for patient movement in real-time, as described herein.

The robot control system can navigate the robot in a number of scenarios. For example, the robot control system can adjust the position of the robot while the robot is still in space (e.g., providing a rigid port to the patient). In some implementations, the robot control system can adjust the position of the robot along one or more axes while an operator of the robot is moving the robot or the instrument down to a target location through a predetermined trajectory. The robot control system can further navigate the robot using the techniques described herein above in connection with FIGS. 4 and 6. For example, the robot control system can generate instructions to adjust the position of the robot in response to a 3D point cloud representing the patient indicates that the patient has moved. In addition, the robot control system can generate instructions to adjust the position of the robot in response to conditions of measured signals from one or more sensors (e.g., the sensors 855). In some implementations, the robot control system can navigate the robot continuously or in near real-time, to closely match the conditions of the surgical environment (e.g., sudden patient movement, instantaneous forces experienced, etc.).

At STEP 904, the robot control system can determine whether a collaborative control condition has been detected. The robot control system can detect a collaborative control condition of the robot based on one or more conditions of the surgical environment. As described herein above, the robot can operate autonomously, semi-autonomously, or manually, where the surgeon has complete control over the position and orientation of the robot and the instrument. Under certain conditions, it is advantageous to initiate collaborative control of the robot, such that the surgeon has total manual control over the position and orientation of the surgical instrument. The robot control system can monitor the conditions of the surgical environment using, for example, information from an image processing system (e.g., the image processing system 100) or the sensors to detect whether a collaborative control condition has been satisfied.

In some implementations, the robot control system can access and monitor 3D point clouds corresponding to the patient via the image processing system 100. As described in greater detail herein above in connection with FIG. 4, the robot control system (which can include any of the functionalities of the robot control system 405) can receive 3D images corresponding to the patient, and identify one or more points in the 3D point cloud over time. By monitoring the positions of the points in the 3D point clouds, the robot control system can determine a change in position of the patient over time. In some cases, it is advantageous to allow the surgeon to have full manual control of the robot and the instrument when patient movement is detected that exceeds one or more thresholds. For example, in some implementations, if the patient moves by a predetermined displacement over a predetermined amount of time (e.g., a relatively large distance in a relatively short amount of time), the robot control system can determine that the control condition is satisfied and generate a signal for the robot control system to initiate collaborative control of the robot.

In some implementations, the robot control system can identify measurements produced by the sensors. For example, to identify measurements from the sensors, the robot control system can perform operations such as those described herein above in connection with the measurement identifier 635 of FIG. 6. For example, in some implementations, one or more of the sensors can be positioned on or coupled to the patient during the surgical procedure. The robot control system can identify measurements form the sensors, for example, to determine an amount of patient movement over time. Using the recorded position values of the patient determined from the measurements of the sensors, the robot control system can determine an amount of movement over time by comparing the current (e.g., from the most recent sensor measurements) position of the patient to the initial conditions of the patient. The robot control system can compare the amount of patient movement to a patient movement condition (e.g., a predetermined threshold of patient movement over time that will trigger collaborative control). The robot control system can continuously or periodically monitor the measurements from the sensors so that patient movement can be monitored in real-time. The robot control system can compare patient movement to the threshold each time it is detected, and generate a signal for the robot control system to initiate collaborative control of the robot if the movement condition has been satisfied.

In some implementations, one or more of the sensors can be positioned at a junction between the robot and the patient. For example, in some implementations, the robot may be secured to the patient (e.g., screwed into a patient's skull, etc.). If the robot control system detects measurements from the force sensors on the robot that indicate patient movement (e.g., that exceeds a threshold, etc.), the robot control system can generate a signal for the robot control system to initiate collaborative control of the robot. In some implementations, one or more of the sensors can be positioned on the instrument, such as on a tip of a needle forming a part of the instrument. The robot control system can detect that measurements from the sensor(s) positioned at the needle tip (e.g., IMU sensors, accelerometers, torque sensors, etc.) indicate that the needle tip has deviated from the predetermined pathway (e.g., the tip has bent or deformed due to external force, etc.). In response, the robot control system can generate a signal for the robot control system to initiate collaborative control of the robot.

In some implementations, the robot control system can detect the collaborative control condition based on signals received from the sensors positioned on the robot or the instrument. For example, in some implementations, the sensors can have forces applied to the robot by a surgeon operating the robot. If the surgeon applies a force that exceeds a predetermined threshold in a predetermined amount of time (e.g., a jerking motion, etc.), this can indicate that the surgeon is attempting to take manual control of the robot. In response, the robot control system can generate a corresponding signal to initiate collaborative control of the robot. The robot control system can also detect collision events throughout the robot from sensors coupled to the robot. For example, if the robot is a robotic arm, such as the robotic arm 310, one or more of the sensors can be positioned at the robotic arm and provide measurements of external forces (e.g., collisions) experienced by the robot. The robot control system can detect any collisions with the robot based on the measurements from the sensors (e.g., measurements exceeding a threshold that indicate unexpected movement, etc.) and generate a signal for the robot control system to initiate collaborative control of the robot.

The robot control system can detect the collaborative control condition based on an error condition in an image-to-patient registration process, such as the image-to-patient process performed by the image registration component 445 described herein above in connection with FIG. 4. As described herein, the robot control system can perform an image-to-patient registration technique to align a 3D image of the patient, which may include indicators of potential targets, and the 3D point cloud captured from the patient. A 3D image, such as a CT scan of the patient's head, can include both a 3D representation of the patient's face and an indication of the biopsy region within the patient's brain. By registering the 3D image of the patient with the real-time 3D point cloud of the patient in the surgical environment, the robot control system can map the 3D image of the patient, and any target indicators, into the same frame of reference as both the 3D point cloud, the tracked position of the robot, and the tracked position of the instrument 430. Doing so allows the robot control system to track the position of the instrument with respect to both the patient and the target regions indicated in the 3D image of the patient. To register the 3D image to the 3D point cloud of the patient, the robot control system can perform an iterative fitting process, such as a RANSAC algorithm and an iterative closest-point algorithm. The robot control system can continuously register the 3D image to the 3D point cloud of the patient. If registration fails (e.g., the fitting algorithm fails to fit the 3D image to the 3D point cloud within a predetermined error threshold, etc.), the robot control system can generate a signal indicating the failure. In response to detecting the failure signal from the robot control system, the robot control system can generate a signal for the robot control system to initiate collaborative control of the robot if the condition has been satisfied.

In addition, a surgeon may manually initiate collaborative control of the robot. As described herein, the robot can include one or more buttons, or may be control through one or more user interfaces, such as a touch screen. In some implementations, a button positioned on the robot (such as one of the buttons 335 described herein in connection with FIG. 3), or an actionable object (e.g., a graphical button, a hyperlink, or other user interface element, etc.) on a user interface can generate a signal to the robot control system to initiate collaborative control. The robot control system can receive a signal indicating an interaction with a button corresponding to the collaborative control condition, and generate a signal for the robot control system to initiate collaborative control of the robot.

At STEP 906, the robot control system can generate instructions to initiate collaborative control of the robot. Upon detecting the signal generated by the control condition detector 840, the robot control system can generate instructions for the robot to provide manual control to a surgeon operating the robot. In some implementations, the robot control system can store an indication of the collaborative control event, and any conditions that caused the collaborative control event to occur, in one or more data structures in the memory of the robot control system. In some implementations, the robot control system can store the collaborative control event in association with a timestamp indicating when the collaborative control event occurred. The robot control system can communicate the instructions, which may be generated using one or more APIs corresponding to the robot using a communications interface. In some implementations, the robot control system can receive an indication to re-initiate automatic navigation of the robot (e.g., via user input from the surgeon, pressing a button, interaction at a user interface 120, etc.), and the robot control system can generate a signal for the robot control system to continue navigating the robot according to a target trajectory.

### E. Real-Time Non-Invasive Surgical Navigation Techniques

The real-time, surface-based registration system as described with respect to FIG. 1, among other figures, can track the location of pre-planned brain targets during a procedure. For example, 3D camera data can be aligned with medical image (e.g., CT or MRI) data and tracked in real-time to track the pre-planned targets. The system can then control the location of a surgical device, such as a robotic device, to orient instruments with respect to the target. Various brain-related procedures typically require a cranial clamp or other device to limit movement of the subject's head, which can make performing the procedures more uncomfortable and time-consuming. However, performing non-invasive procedures without a cranial clamp necessitates constant adjustments to account for patient movement. For example, in transcranial magnetic stimulation (TMS), the practitioner specifically targets an area of the cortex to stimulate neurons. Current practice approximates the target region by marking the patient. Without precise targeting, sudden movement may lead to stimulation in undesired cranial regions with uncertain side effects. In addition, the skull can create large diffraction of signals such as TMS or ultrasound signals, further complicating accurate therapy delivery.

The present solution can map the patient's cortex prior to delivering therapy via CT scans. This allows for internal navigation of the patient's morphology to precisely target locations of interest. Once therapy delivery begins, the present solution can automatically stop emitting energy (or otherwise adjust the amount of emitted energy) from the therapeutic device when the system detects a wrong registration, the patient moves too quickly, etc. It can also automatically stop delivering energy once a therapy condition is satisfied, such as if a predefined therapeutic threshold is achieved. The present solution can use data such as a patient's morphology and locations of interest for the therapy. Additionally, the present solution can combine focal steering in therapy devices to achieve fine adjustments of a focal point.

For applications that require the device to be in contact with the patient's skin, the present solution can combine torque sensing with the surface-based registration system. The present solution can utilize tracking data of the instrument as well as data collected from 3D image processing to monitor surface contact. This creates a condition in which the device can stay on-target while in contact with a surface of the patient, such as skin or the scalp, and can apply a predefined amount of force or range of force to the surface. In the event of slight patient movements, the present solution can adjust and stay in contact with the target location with the same predefined amount of force. This can allow for precise therapy delivery as well as patient comfort since therapy sessions can last for hours.

FIG. 10 depicts an example of a system 1000. The system 1000 can incorporate features of various systems and devices described herein, including but not limited to the system 200 described with reference to FIG. 2. The system 1000 can be used to perform non-invasive procedures, particularly real-time non-invasive procedures on or around the head of a subject, including to deliver therapy to the brain of the subject.

The system 1000 can include features of the image processing system 1000 described with reference to FIGS. 1 and 2. The image processing system 1000 can include one or more image capture devices 1002, which may be similar to and include any of the structure and functionality of the image capture devices 1002 described in connection with FIGS. 1 and 2. Each of the image capture devices 1002 can include one or more lenses 1003, which may be similar to and include any of the structure and functionality of the lenses 1003 in connection with FIGS. 1 and 2. The lenses 1003 can receive light indicative of an image. The image capture devices 1002 can include sensor circuitry that can detect the light received via the one or more lenses 1003 and generate images 1007 based on the received light. The images 1007 can be similar to the images 1007 described in connection with FIGS. 1 and 2.

The image processing system 1000 can include communications circuitry 216. The communications circuitry 1016 can implement features of computing device 1200 described with reference to FIGS. 12A and 12B. The communications circuitry 1016 can be similar to, and can incorporate any of the structure or functionality as, the communications circuitry 1016 described in connection with FIGS. 1 and 2.

The image processing system 1000 can include one or more tracking sensors, such as IR sensors 1018 and image capture devices 1002. The IR sensors 1018 can be similar to, and can include any of the structure or functionality of, the IR sensors 220 described in connection with FIGS. 1 and 2. The IR sensors 1018 can detect IR signals from various devices in an environment around the image processing system 100. The IR sensors 1018 can be communicatively coupled to the other components of the image processing system 100, such that the components of the image processing system 1000 can utilize the IR signals in appropriate operations in the image processing pipeline.

The image processing system 1000 can include a surgical instrument 1004. The surgical instrument 1004 can deliver therapy to the subject and its relative location 1008 is determined by images 1007 captured by the image capture devices 1002. A parameter 1012 signifies the amount of energy that has been delivered and is processed by the processing circuitry 1014. The processing circuitry 1014 can be similar to, and can include any of the structure or functionality of, the processing circuitry 1014 described herein. Likewise, the image processing system 1000 of FIG. 10 may be implemented in addition to or as an alternative to the image processing system 100 of FIGS. 1 and 2, to perform any of the functionality described herein. The surgical instrument 1004 can be, for example, a focused ultrasound device, transducer, magnetic coil, etc.

Two or more capture devices 1002 can capture 3D images of the subject for accuracy and overall resolution. The processing circuitry 1014 can extract 3D data from each data point in the images 1007 received from the image capture devices 1002 and generate a point cloud corresponding to each capture device 1002. In some implementations, the processing circuitry 1014 can down-sample data points to reduce the overall size of images 1007 without significantly affecting the accuracy of further processing steps, improving the image processing.

The processing circuitry 1014 can select one of the point clouds to act as a reference frame for the alignment of any of the other point clouds. Selecting the reference frame can include retrieving color data assigned to one or more of the first set of data points of the first point cloud where the processing circuitry 1014 can extract the color data. Selecting the reference frame can also include determining the most illuminated point cloud, least uniformly illuminated, or the processing circuitry 1014 can arbitrarily choose a reference frame of a point cloud as the reference frame.

The processing circuitry 1014 can determine a transformation data structure such that when each matrix is applied to a respective point cloud, the features of the transformed point cloud will align with similar features in the reference frame point cloud. The transformation matrices include transformation values that indicate a change in position or rotation of the points in the point cloud to be transformed.

Using the information from the global scene, the processing circuitry 1014 can determine a location of interest within the first reference frame related to the first point cloud and the 3D medical image. If a location of interest is detected, the processing circuitry 1014 ca generate a highlighted region within the display data rendered in the user interface 1020. The user interface 1020 can be similar to, and may include any of the structure and functionality of, the user interface 120 described in connection with FIGS. 1 and 2. This location of interest can be input by a medical professional for non-invasive applications of the image processing system 1000.

The processing circuitry 1014 can be configured to determine a distance of the subject represented in the 3D medical image from the image capture device 1002 responsible at least in part for generating the first point cloud. If there is a reference object or marker in the global scene that has a known distance or length, the processing circuitry 1014 can use the known distance or length to determine the distance from the image capture devices 1002 to other features in the global scene. The processing circuitry 1014 can determine an average location of the subject using features of the subject in the global point cloud that correspond to the features in the 3D medical image.

The processing circuitry 1014 can use this same method to determine an average location of the surgical instrument 1004. The computer-generated model of the surgical instrument 1004 can be registered by the processing circuitry 1014, and matched with the 3D image data collected by the image capture devices 1002. The processing circuitry 1014 can use known distances or lengths to calculate different dimensions or parameters of the global scene point cloud to determine the distance of the image capture devices 1002 to the surgical instrument 1004. Using the features of the subject and the relative location 1008, the processing circuitry can determine the distance of the surgical instrument 1004 to the subject by processing the tracking data gathered by the IR sensors 1018 and the reference frame aligned with the 3D image data captured by the image capture devices 1002. The relative location 1008 of the surgical instrument 1004 can be continuously tracked by the image capture devices 1002 in parallel with the IR sensors 1018 with tracking data sent to the processing circuitry 1014.

The surgical instrument 1004 can deliver procedure to the location of interest. The processing circuitry 1014 can communicate with the surgical instrument 1004 through the communications circuitry 1016. The processing circuitry 1014 can track the total amount of energy being delivered to the location of interest through the parameter 1012. The processing circuitry 1014 can also track the total amount of time of the energy being delivered. The processing circuitry 1014 can terminate, reduce amount of energy being output, or otherwise change parameters of energy delivery of the procedure to the location of interest if parameter 1012 is satisfied or if location of interest is no longer aligned with the surgical instrument.

In some implementations, the processing circuitry 1014 can communicate through the communications circuitry 1016 display data to the user interface 1020 the internal mapping of the subject the surgical instrument 1004 is targeting. The processing circuitry 1014 can use 3D medical image data (e.g. CT, MRI) and align the data to the global scene to generate the display data. For example, the surgical instrument 1004 could be a transducer that targets certain internal locations of the subject.

The processing circuitry 1014 can calculate and provide up-to-date information on the relative location 1008 to the location of interest through the IR sensors 1018. The processing circuitry 1014 registers the initial alignment of the location of interest and relative location 1008 through tracking information received from the IR sensors 1018 and 3D image data from the image capture devices 1002. If the processing circuitry 1014 detects movement with a velocity below the allowed velocity threshold of the location of interest, the processing circuitry 1014 will generate movement instructions to the surgical instrument 1004 to re-align with the location of interest. If the processing circuitry 1014 detects movement with a distance below the allowed distance threshold of the location of interest, the processing circuitry 1014 will generate movement instructions to the surgical instrument 1004 to re-align with the location of interest. If the processing circuitry 1014 detects movements with a velocity above the allowed velocity threshold of the location of interest, the processing circuitry 1014 will transmit termination instructions through the communication circuitry 1016. If the processing circuitry 1014 detects movement with a distance above the allowed distance threshold of the location of interest, the processing circuitry 1014 will transmit termination instructions through the communication circuitry 1016.

In some implementations, the surgical instrument 1004 can be in contact with the subject. The processing circuitry 1014 registers the global scene and the relative location 1008 of the surgical instrument 1004 to the location of interest. The processing circuitry 1014 can receive information from sensors, such as IR sensors 1018, and processes lateral and rotational movement of the location of the interest. The processing circuitry 1014 can generate movement instructions to keep the surgical instrument 1004 in contact with the subject with a predetermined amount of force. The processing circuitry 1014 can generate movement instructions to keep the surgical instrument 1004 in contact with the subject with a predetermined amount of torque. The processing circuitry 1014 transmits movement instructions through the communication circuitry 1016 for the system 1000 to include torque sensing in its non-invasive surgical navigation.

In some implementations, the surgical instrument 1004 can output an ultrasonic signal for therapy delivery purposes. For example, in focused ultrasound therapy, the surgical instrument 1004 can deliver ultrasounds to location of interests and specifically opens the blood-brain barrier to non-invasively deliver drug therapy. In some implementations, the surgical instrument 1004 can include a plurality of ultrasound transmitter elements, such as transducers, arranged in an array. In some implementations, the surgical instrument 1004 can perform beamforming using the plurality of ultrasound transmitter elements to superpose wavefronts to create plane waves. In some implementations, the surgical instrument 1004 can control various parameters like wave frequency to control and steer the outputted ultrasonic signal. The processing circuitry 1014 can control the surgical instrument 1004 to perform focal steering of the ultrasound beam, such as to control phased array operation or other operations of the surgical instrument 1004 to control at least one of a position and a direction of the ultrasound beam based on at least one of the tracking data of the surgical instrument 1004 or a target parameter of the procedure being performed using the ultrasound beam.

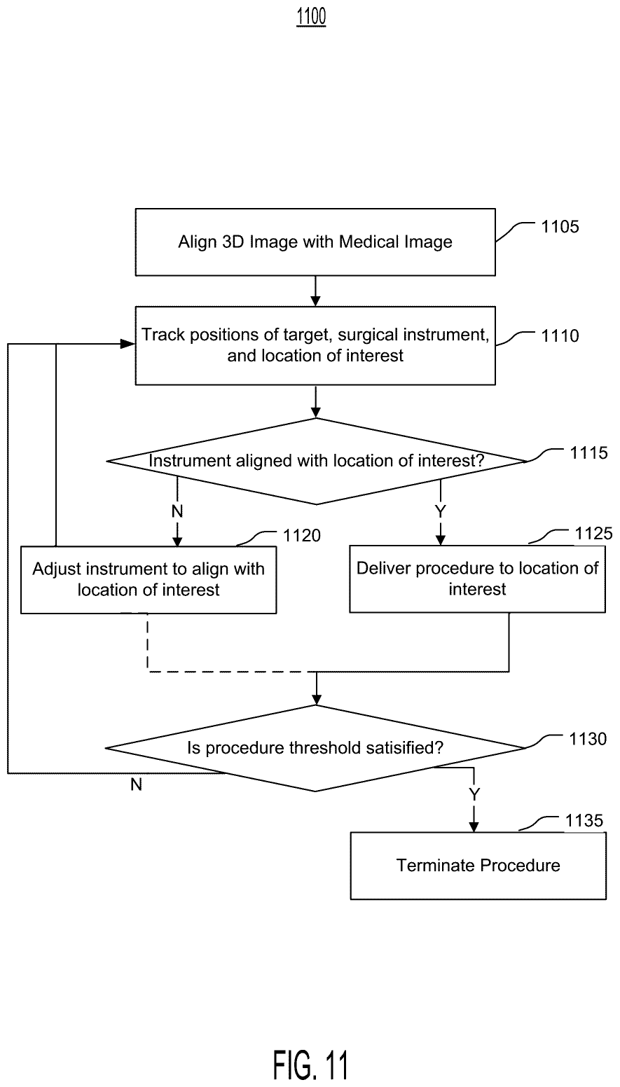

FIG. 11 depicts a method 1100 for real-time non-invasive surgical navigation to facilitate delivering a procedure to a location of interest on a subject, such as on a head of the subject. The method 1100 can be performed using any of the computing systems described herein, including the image processing system 100 of FIG. 1, the image processing system 1000 of FIG. 10, the robot control system 405 of FIG. 4, the robot control system 605 of FIG. 6, the robot control system 805 of FIG. 8, or the computer system 1200 of FIGS. 12A and 12B. It will be appreciated that the steps of the method 1100 may be completed in any order, including the performance of additional steps or the omission of certain steps, to achieve desired results.

The method 1100 can be performed using a magnetic coil for transcranial magnetic stimulation, a high powered ultrasound, or other surgical instruments use for non-invasive cranial procedures. For example, the method 1100 can be performed to maintain alignment between real-time 3D image data of the subject and model data of the subject (e.g., model data from 3D medical image data, such as CT or MRI data), control a surgical instrument to apply a procedure to the subject based on a location of interest associated with the model data and the alignment with the real-time 3D image data of the subject, monitor the procedure as well as the relative positions of the surgical instrument and the subject and, in some implementations, force (e.g., torque) data indicative of contact between the surgical instrument and the subject, and control how the procedure is performed, including terminating the procedure, adjusting energy or other outputs of the surgical instrument, and/or moving the surgical instrument, responsive to the monitoring. This can enable the procedure to be performed more accurately and with less likelihood of off-target delivery of therapy, such as magnetic or ultrasound signals, to the subject.

At 1105, a 3D image is positioned relative to a medical image of a subject. The medical image can include CT or MRI image data, which may be used as a model of the subject. The medical image can include 3D medical image data. The 3D image may be a point cloud captured using one or more image capture devices (e.g., the image capture devices 1002 of FIG. 10, etc.). The point cloud can depict a patient in a surgical environment. One capture of a 3D point cloud (sometimes referred to as a “3D image”) may be referred to as a “frame” or a “point cloud frame,” which can correspond to a single capture by the image capture devices. The point cloud and/or frame of reference can be generated from the medical image or from the 3D image captured using the image capture devices. The 3D image can be positioned relative to the medical image by registering or aligning the medical image with the 3D image using various methods described herein. The positioning can be updated periodically as 3D image data is received, e.g. from sequential captures of the 3D image using a 3D camera or other image capture device as described herein.

The 3D image aligning process can be implanted using an image-to-patient registration technique to align a 3D image of the patient, which may include indicators of potential targets, and the 3D point cloud captured from the patient in the surgical environment. A 3D medical image, such as a CT scan of the patient's head, can include both a 3D representation of the patient's face and an indication of a target region within the patient's brain or another location of the patient's anatomy. By registering the 3D image of the patient with the real-time 3D point cloud of the patient in the surgical environment, the 3D image of the patient can be mapped to the medical image, along with any target indicators, in the same frame of reference as both the real-time 3D point cloud of the patient and any instruments in the environment surrounding the patient. Doing so can allow for accurate tracking of the position of one or more instruments that may be utilized in procedures involving the patient, with respect to both the patient and the target regions indicated in the 3D image of the patient. To register the 3D image to the 3D point cloud of the patient, an iterative fitting process, such as a RANSAC algorithm or an iterative closest-point algorithm can be performed. In doing so, the 3D image can be continuously (or periodically, in response patient or instrument movement, etc.) registered to the 3D point cloud of the patient. If registration fails (e.g., the fitting algorithm fails to fit the 3D image to the 3D point cloud within a predetermined error threshold, etc.), a signal can be generated that indicates the failure.

At 1110, at least one of a surgical instrument and the subject can be tracked, such as to track positions of the surgical instrument and/or the subject relative to the frame of reference or specific locations in the frame of reference. The surgical instrument and/or subject can be tracked using various sensors, such as image capture devices (including a 3D camera used to detect the 3D image), infrared sensors, torque or force sensors, or various combinations thereof. The surgical instrument and subject can be tracked periodically, such as to periodically update the locations of the surgical instrument and subject relative to the model used to represent the subject (and surgical instrument).

Tracking can include determining a position of a surgical robot, which may be equipped or coupled to a non-invasive instrument, within the same frame of reference as the 3D point cloud of the patient in the surgical environment. As described herein, the image capture devices can capture images of a patient in a surgical environment. The image capture devices can have a predetermined pose within the surgical environment relative to other sensors, such as IR sensors (e.g., the IR sensors 1018). As described herein above, the IR sensors can be IR cameras that capture IR wavelengths of light. One or more tracking techniques, such as IR tracking techniques, may be utilized to determine the position of the robot. The robot can include one or more markers or indicators on its surface, such as IR indicators. The IR sensors can be used to determine the relative position of the robot. In some implementations, the orientation (e.g., the pose) of the robot can be determined by performing similar techniques. Because the sensors used to track the position of the robot are a known distance from the image capture devices, the position of the markers detected by the IR sensors can be mapped to the same frame of reference as the 3D point cloud captured by the image capture devices.

In some implementations, the image capture devices may be used to determine the position of the robot. For example, the robot can include one or more graphical indicators, such as bright distinct colors or QR codes, among others. In addition to capturing 3D point clouds of the patient, the image capture devices can capture images of the surgical environment, and image analysis techniques can be performed to determine the position of the surgical robot in the images based on the detected positions of the indicators in the images. The position and orientation of the surgical robot can be computed periodically, for example, in real-time or near real-time, enabling the robot control system to track the movement of the robot over time. In some implementations, a calibration procedure can be performed to establish the unified frame of reference between the 3D point clouds and the indicators coupled to the surgical robot. The calibration procedure can include identifying the position of the robot with respect to global indicators positioned in the surgical environment. The position of the robot can be stored in one or more data structures of a computer-readable memory, such that the real-time or near real-time position of the robot can be accessed various components described herein.

Tracking the position of the robot can include tracking the position of an instrument (e.g., the surgical instrument 1004) coupled to the robot. As described herein above, the instrument can include its own indicators that are coupled to the instrument or a bracket/connector coupling the instrument to the robot. The position and orientation of the instrument, as well as the robot, can be tracked in real-time or near real-time using techniques similar to those described herein. The position and orientation of the instrument can be stored in the computer-readable memory, for example, in association a respective timestamp.

Additionally, patient location and movement can be accurately determined, and signals can be generated to adjust the position of the robot accordingly. To do so, previous positions of the 3D point cloud representing the patient can be compared to the positions of current or new 3D point clouds captured by the image capture devices. In some implementations, at least two sets of 3D point clouds can be maintained (e.g., stored, updated, etc.): one from a previously captured frame and another from a currently captured (e.g., most-recently captured) frame. The comparison can be a distance (e.g., a Euclidean distance, etc.) in the frame of reference of the 3D point clouds.

Iterative computations can be performed to determine which 3D points in a first 3D point cloud (e.g., the previous frame) correspond to 3D points in a second 3D point cloud (e.g., the current frame). For example, an iterative ICP algorithm or a RANSAC fitting technique can be performed to approximate which points in the current frame correspond to the points in the previous frame. Distances between the corresponding points can then be determined. In some implementations, to improve computational performance, the robot control system can compare a subset of the points (e.g., by performing a down-sampling technique on the point clouds, etc.). For example, after finding the point correspondences, a subset of the matching point pairs can be selected between each point cloud to compare (e.g., determine a distance between in space, etc.). In some implementations, to determine the movement of the patient in the surgical environment, an average movement of the 3D points between frames can be calculated. Similar techniques can be used to calculate the position or movement of the target location of the patient's anatomy within the surgical environment based on the image registration techniques described herein. Additionally, and as described herein, sensor data from one or more torque sensors may be utilized to detect movement or position of the patient, the surgical robot, the instrument, or the target location of the patient's anatomy within the surgical environment.

At 1115, alignment of the surgical instrument with a location of interest can be evaluated. The location of interest (sometimes referred to as a target location) can be a location on the subject, such as a location on a skull of the subject that is related to a procedure. The location of interest can be identified in the medical image or the 3D image data, such as based on being marked by a user (e.g., surgeon) in a procedure plan or other information associate with the medical image. For example, the location of interest can be a site on the head of the subject at which ultrasound, magnetic signals, or other non-invasive signals, are to be applied. The surgical instrument can be used to perform various procedures (e.g., including invasive procedures and non-invasive procedures) on the subject at the location of interest. Alignment of the surgical instrument with the location of interest can be evaluated based on tracking data from the tracking of the surgical instrument and the subject, and can be evaluated based on at least one of a detected distance between the surgical instrument and the location of interest as compared with a target distance and an orientation of the surgical instrument as compared with a target orientation (e.g., angle at which the surgical instrument should be positioned relative to the head of the subject to facilitate effective therapy delivery). An output of the evaluation of the alignment can include an indication as to whether the surgical instrument is or is not aligned with the location of interest, such as by determining that the surgical instrument is (or is not) within a target distance or range of distances from the location of interest and is oriented at an angle or within a range of angles relative to a surface of the subject at the location of interest. Techniques similar to those described in Sections A through D can be performed to determine the position and orientation of the surgical instrument relative to the target location (and target orientation for the procedure being performed). If the instrument is aligned with the location of interest and the target orientation, step 1125 of the method 1100 can be executed. Otherwise, if the instrument is not aligned with the location of interest or not at the target orientation (or within a predetermined tolerance range of either), step 1120 of the method 1100 can be executed.

At 1120, responsive to detecting that the surgical instrument is not aligned with the location of interest, movement instructions can be transmitted to at least one of the surgical instrument or a robotic device (e.g., robotic arm) coupled with the surgical instrument to adjust the pose of the surgical instrument to align the surgical instrument with the location of interest. The movement instructions can be generated and transmitted periodically responsive to periodic evaluation of the alignment.

As described herein, the position or orientation of the robot can be adjusted when the robot (or a controller coupled to the robot) executes or interprets instructions or signals indicating a target location or target orientation for the instrument. For example, the robot can execute such instructions that indicate a target location or target orientation for the instrument, and actuate various movable components in the robot to move the instrument to the target location and target orientation. The robot control system can navigate the robot according to the movement of the patient or the target location on the patient's anatomy, such that the robot or the instrument is aligned with a target location or target pathway for the procedure, even when the patient is unsecured. In some implementations, the target location or the target pathway can be specified as part of the 3D image (e.g., the CT scan or the MRI scan, etc.) that is registered to the real-time 3D point cloud representing the patient in the frame of reference of the image capture devices. As the location of the instrument and the robot are also mapped within the same frame of reference, an accurate measure of the distance between the instrument and the can be determined. An offset may be added to this distance based on known attributes of the instrument, for example, to approximate the location of a predetermined portion of the instrument (e.g., the tip or tool-end) within the surgical environment.

When it is determined that the surgical instrument is misaligned with the target location or the target orientation, instructions can be generated to move or re-orient the robot according to align the instrument with the target location or the target orientation. For example, from the CT scan or from user input, the instrument may be aligned with a target pathway or target location in the surgical environment to carry out the procedure. If the patient moves during the surgical procedure, instructions can be generated, for example, using one or more APIs of the robot to move the instrument in-step with the detected patient movement or the detected misalignment. In one example, if patient movement of 2 centimeters to the left of a target pathway is detected, the robot control system can adjust the position of the robot the same 2 centimeters to the left, according to the target pathway. In some implementations, adjustments to the instrument's position or orientation can be made while navigating the robot along a predetermined trajectory (e.g., a pathway into the skull of the patient to reach a target location on the patient's anatomy, etc.). For example, the robot may be navigated left or right according to patient movement or to realign the instrument with the target position or orientation, while also navigating the instrument downward along a predetermined trajectory to the target location on the patient's anatomy. This provides an improvement over other robotic implementations for non-invasive procedures that do not track and compensate for patient movement, as any patient movement during a procedure could result in patient harm. By moving the instrument in accordance with patient movement, unintended collisions or interference with other parts of the patient are mitigated, as the predetermined target pathway may be followed more exactly.

The adjustments to the position or orientation of the robot or instrument can be performed iteratively, or periodically and multiple times per second, to compensate for sudden patient movement or other misalignment of the surgical instrument. As described herein, patient movement and the position of the instrument within the surgical environment can be calculated periodically (e.g., according to the capture rate of the image capture devices, etc.). Each time a new frame is captured by the image capture devices, it can be determined whether the change in the position of the patient satisfies a threshold (e.g., a predetermined amount of movement relative to a previous frame, to a patient position at the start of the procedure, or to the position of the instrument, etc.). The position or orientation of the robot can be adjusted such that the instrument is aligned with the predetermined pathway (e.g., a predetermined trajectory) relative to the detected change in the position of the patient. The change in the position of the trajectory or pathway can be determined based on a change in position or orientation of one or more target indicators in the 3D images (e.g., the CT scan or MRI scan, etc.) that are registered to the patient in real-time. In some implementations, the 3D images can be modified to indicate the pathway or trajectory to the selected targets. Likewise, in implementations where multiple targets or pathways (or segments of pathways) are present, the surgeon may select one or more pathways along which the robot should navigate the instrument via user input (e.g., button selections, selections by touch screen, etc.). The robot can be navigated along the selected pathways while compensating for patient movement or misalignment of the surgical instrument in real-time, as described herein. After aligning the instrument at the target location and orientation, the position and orientation of the instrument can be continuously tracked at step 1110. In some implementations, step 1120 can be executed in parallel with step 1125 (e.g., enabling the simultaneous re-alignment of the surgical instrument and application of the instrument output to the target location). In such implementations, the method 1100 may include the execution of step 1130 during the execution of steps 1110 through 1125.

At 1125, the surgical instrument can be controlled to apply a procedure, such as to deliver TMS or FUS therapy to the location of interest. The surgical instrument can be controlled to apply the procedure responsive to detecting that the surgical instrument is aligned with the location of interest; the procedure can be adjusted, paused, or terminated responsive to detecting that the surgical instrument is not aligned with the location of interest. Using the APIs of the robot, or of the instrument, instructions can be generated to activate the instrument to apply the desired procedure. In some implementations, a manual input can be provided (e.g., by a surgeon, etc.) to activate the instrument to apply the procedure. In an implementation where the procedure is automatically applied (e.g., responsive to detecting that the instrument is aligned at the target location and orientation), instructions for the procedure can be accessed or retrieved to determine the duration and intensity of the signals to be applied. The procedure can be a non-invasive procedure. In some implementations, the duration, intensity, and type of instrument output can be pre-selected by a surgeon or from a database of procedures. In some implementations, several target locations can be selected, each with a corresponding duration, intensity, and type of instrument output. The various steps of the method 1100 can be applied to each of the several target locations to achieve the desired outputs to the complete the non-invasive procedure.

At 1130, performance of the procedure is evaluated. For example, various parameters of the procedure, such as duration, instantaneous, average, and/or total energy or power (e.g., of a delivered beam or signal), as well as responses of the subject, such as physiological or biological responses (e.g., heart rate, breathing rate, temperature, skin conductance, brain wave activity, or various other parameters detected by various sensors) can be evaluated by being compared with respective thresholds. Such responses can be captured from sensors coupled to the patient during the procedure. In some implementations, delivery of the therapy can be adjusted responsive to the evaluation, such as to increase or decrease power, energy, frequency, or other parameters of the magnetic field or ultrasound signal being used to perform the therapy (in addition to adjusting the pose of the surgical instrument). The respective thresholds can be provided, for example, by a surgeon prior to the procedure. Instructions to adjust the parameters of the procedure can be generated using similar techniques to those described herein. If the surgical procedure threshold(s) are satisfied, the method 1100 can proceed to step 1135. If the surgical procedure threshold(s) are not satisfied, the method 1100 can return to step 1110, which may be executed in parallel with step 1125 to reposition or reorient the surgical instrument while the procedure is applied to the target location of the patient.

At 1135, responsive to the evaluation of the performance satisfying a termination condition (e.g., sufficient duration and/or total energy delivery), the surgical instrument can caused to discontinue the procedure. In some implementations, the termination of the procedure can include generating instructions to modify the position or orientation of the robot to a default position or orientation that separates the instrument from the anatomy of the patient. These instructions can be generated using techniques similar to those described herein. Generating the instructions can include generating instructions to guide the instrument along a predetermined trajectory to safely separate the instrument from the anatomy of the patient. In the case of an invasive procedure, this can include following a trajectory (e.g., which may be predetermined or determined at part based on the current position of the robot) that safely removes the instrument from within the anatomy of the patient. Similar techniques can be performed to remove the instrument from the surface of the patient's anatomy in a non-invasive procedure. In some implementations, an alert can be generated that indicates the procedure is complete, and the surgeon can be prompted to manually control the robot to remove the instrument from the patient's anatomy.

### F. Computing Environment

FIGS. 12A and 12B depict block diagrams of a computing device 1200. As shown in FIGS. 12A and 12B, each computing device 1200 includes a central processing unit 1221, and a main memory unit 1222. As shown in FIG. 12A, a computing device 1200 can include a storage device 1228, an installation device 1216, a network interface 1218, an I/O controller 1223, display devices 1224a-1224n, a keyboard 1226 and a pointing device 1227, e.g. a mouse. The storage device 1228 can include, without limitation, an operating system, software, and software of the image processing system 100, the robot system 300, the robot control system 405, the robot control system 605, the robot control system 805, or the image processing system 1000. As shown in FIG. 12B, each computing device 1200 can also include additional optional elements, e.g. a memory port 1203, a bridge 1270, one or more input/output devices 1230a-1230n (generally referred to using reference numeral 1230), and a cache memory 1240 in communication with the central processing unit 1221.

The central processing unit 1221 is any logic circuitry that responds to and processes instructions fetched from the main memory unit 1222. In many embodiments, the central processing unit 1221 is provided by a microprocessor unit, e.g.: those manufactured by Intel Corporation of Mountain View, California; those manufactured by Motorola Corporation of Schaumburg, Illinois; the ARM processor (from, e.g., ARM Holdings and manufactured by ST, TI, ATMEL, etc.) and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California; or field programmable gate arrays (“FPGAs”) from Altera in San Jose, CA, Intel Corporation, Xilinx in San Jose, CA, or MicroSemi in Aliso Viejo, CA, etc. The computing device 1200 can be based on any of these processors, or any other processor capable of operating as described herein. The central processing unit 1221 can utilize instruction level parallelism, thread level parallelism, different levels of cache, and multi-core processors. A multi-core processor can include two or more processing units on a single computing component. Examples of multi-core processors include the AMD PHENOM IIX2, INTEL CORE i5, INTEL CORE i7, and INTEL CORE i9.

Main memory unit 1222 can include one or more memory chips capable of storing data and allowing any storage location to be directly accessed by the microprocessor 1221. Main memory unit 1222 can be volatile and faster than storage 1228 memory. Main memory units 1222 can be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 1222 or the storage 1228 can be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 1222 can be based on any of the above described memory chips, or any other available memory chips capable of operating as described herein. In the embodiment shown in FIG. 12A, the processor 1221 communicates with main memory 1222 via a system bus 1250 (described in more detail below). FIG. 12B depicts an embodiment of a computing device 1200 in which the processor communicates directly with main memory 1222 via a memory port 1203. For example, in FIG. 12B the main memory 1222 can be DRDRAM.

FIG. 12B depicts an embodiment in which the main processor 1221 communicates directly with cache memory 1240 via a secondary bus, sometimes referred to as a backside bus. In other embodiments, the main processor 1221 communicates with cache memory 1240 using the system bus 1250. Cache memory 1240 typically has a faster response time than main memory 1222 and is typically provided by SRAM, BSRAM, or EDRAM. In the embodiment shown in FIG. 12B, the processor 1221 communicates with various I/O devices 1230 via a local system bus 1250. Various buses can be used to connect the central processing unit 1221 to any of the I/O devices 1230, including a PCI bus, a PCI-X bus, or a PCI-Express bus, or a NuBus. For embodiments in which the I/O device is a video display 1224, the processor 1221 can use an Advanced Graphics Port (AGP) to communicate with the display 1224 or the I/O controller 1223 for the display 1224. FIG. 12B depicts an embodiment of a computer 1200 in which the main processor 1221 communicates directly with I/O device 1230b or other processors 1221 via HYPERTRANSPORT, RAPIDIO, or INFINIBAND communications technology. FIG. 12B also depicts an embodiment in which local busses and direct communication are mixed: the processor 1221 communicates with I/O device 1230a using a local-interconnect bus while communicating with I/O device 1230b directly.

A wide variety of I/O devices 1230a-1230n can be present in the computing device 1200. Input devices can include keyboards, mice, trackpads, trackballs, touchpads, touch mice, multi-touch touchpads and touch mice, microphones (analog or MEMS), multi-array microphones, drawing tablets, cameras, single-lens reflex camera (SLR), digital SLR (DSLR), CMOS sensors, CCDs, accelerometers, inertial measurement units, infrared optical sensors, pressure sensors, magnetometer sensors, angular rate sensors, depth sensors, proximity sensors, ambient light sensors, gyroscopic sensors, or other sensors. Output devices can include video displays, graphical displays, speakers, headphones, inkjet printers, laser printers, and 3D printers. Devices 1230a-1230n can include a combination of multiple input or output devices, including, e.g., Microsoft KINECT, Nintendo Wiimote for the WII, Nintendo WII U GAMEPAD, or Apple IPHONE. Some devices 1230a-1230n can allow gesture recognition inputs through combining some of the inputs and outputs. Some devices 1230a-1230n provide for facial recognition which can be utilized as an input for different purposes including authentication and other commands. Some devices 1230a-1230n provide for voice recognition and inputs, including, e.g., Microsoft KINECT, SIRI for IPHONE by Apple, Google Now, or Google Voice Search.

Additional devices 1230a-1230n have both input and output capabilities, including, e.g., haptic feedback devices, touchscreen displays, or multi-touch displays. Touchscreen, multi-touch displays, touchpads, touch mice, or other touch sensing devices can use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices can allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, can have larger surfaces, such as on a table-top or on a wall, and can also interact with other electronic devices. Some I/O devices 1230a-1230n, display devices 1224a-1224n or group of devices can be augmented reality devices. The I/O devices can be controlled by an I/O controller 1223 as shown in FIG. 12A. The I/O controller 1223 can control one or more I/O devices, such as, e.g., a keyboard 126 and a pointing device 1227, e.g., a mouse or optical pen. Furthermore, an I/O device can also provide storage and/or an installation medium 1216 for the computing device 1200. In other embodiments, the computing device 1200 can provide USB connections (not shown) to receive handheld USB storage devices. In further embodiments, an I/O device 1230 can be a bridge 1270 between the system bus 1250 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 1224a-1224n can be connected to I/O controller 1223. Display devices 1224a-1224n can include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays can use, e.g. stereoscopy, polarization filters, active shutters, or autostereoscopy. Display devices 1224a-1224n can also be a head-mounted display (HMD). In some embodiments, display devices 1224a-1224n or the corresponding I/O controllers 1223 can be controlled through or have hardware support for OPENGL or DIRECTX API or other graphics libraries.

In some embodiments, the computing device 1200 can include or connect to multiple display devices 1224a-1224n, which each can be of the same or different type and/or form. As such, any of the I/O devices 1230a-1230n and/or the I/O controller 1223 can include any type and/or form of suitable hardware, software, or combination of hardware and software to support, enable or provide for the connection and use of multiple display devices 1224a-1224n by the computing device 1200. For example, the computing device 1200 can include any type and/or form of video adapter, video card, driver, and/or library to interface, communicate, connect, or otherwise use the display devices 1224a-1224n. In one embodiment, a video adapter can include multiple connectors to interface to multiple display devices 1224a-1224n. In other embodiments, the computing device 1200 can include multiple video adapters, with each video adapter connected to one or more of the display devices 1224a-1224n. In some embodiments, any portion of the operating system of the computing device 1200 can be configured for using multiple displays 1224a-1224n. In other embodiments, one or more of the display devices 1224a-1224n can be provided by one or more other computing devices 1200a or 1200b connected to the computing device 1200, via the network 1240. In some embodiments software can be designed and constructed to use another computer's display device as a second display device 1224a for the computing device 1200. For example, in one embodiment, an Apple iPad can connect to a computing device 1200 and use the display of the device 1200 as an additional display screen that can be used as an extended desktop. One ordinarily skilled in the art will recognize and appreciate the various ways and embodiments that a computing device 1200 can be configured to have multiple display devices 1224a-1224n.

Referring again to FIG. 12A, the computing device 1200 can comprise a storage device 1228 (e.g. one or more hard disk drives or redundant arrays of independent disks) for storing an operating system or other related software, and for storing application software programs such as any program related to the software for the image processing system 100, the robot system 300, the robot control system 405, the robot control system 605, the robot control system 805, or the image processing system 1000. Examples of storage device 1228 include, e.g., hard disk drive (HDD); optical drive including CD drive, DVD drive, or BLU-RAY drive; solid-state drive (SSD); USB flash drive; or any other device suitable for storing data. Some storage devices 1228 can include multiple volatile and non-volatile memories, including, e.g., solid state hybrid drives that combine hard disks with solid state cache. Some storage device 1228 can be non-volatile, mutable, or read-only. Some storage device 1228 can be internal and connect to the computing device 1200 via a bus 1250. Some storage device 1228 can be external and connect to the computing device 1200 via an I/O device 1230 that provides an external bus. Some storage device 1228 can connect to the computing device 1200 via the network interface 1218 over a network, including, e.g., the Remote Disk for MACBOOK AIR by Apple. Some client devices 1200 may not require a non-volatile storage device 1228 and can be thin clients or zero clients 202. Some storage device 1228 can also be used as an installation device 1216, and can be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Computing device 1200 can also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc.

Furthermore, the computing device 1200 can include a network interface 1218 to interface to the network 1240 through a variety of connections including, but not limited to, standard telephone lines LAN or WAN links (e.g., 802.11, T1, T3, Gigabit Ethernet, or Infiniband), broadband connections (e.g., ISDN, Frame Relay, ATM, Gigabit Ethernet, Ethernet-over-SONET, ADSL, VDSL, BPON, GPON, or fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax, and direct asynchronous connections). In one embodiment, the computing device 1200 communicates with other computing devices 1200 via any type and/or form of gateway or tunneling protocol e.g. Secure Socket Layer (SSL), Transport Layer Security (TLS), or the Citrix Gateway Protocol manufactured by Citrix Systems, Inc. of Ft. Lauderdale, Florida. The network interface 1218 can comprise a built-in network adapter, network interface card, PCMCIA network card, EXPRESSCARD network card, card bus network adapter, wireless network adapter, USB network adapter, modem, or any other device suitable for interfacing the computing device 1200 to any type of network capable of communication and performing the operations described herein.

A computing device 1200 of the sort depicted in FIG. 12A can operate under the control of an operating system, which controls scheduling of tasks and access to system resources. The computing device 1200 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device 1200 and performing the operations described herein. Typical operating systems include, but are not limited to: WINDOWS 2000′, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, can be used on zero clients or thin clients, including, e.g., CHROMEBOOKS.

The computer system 1200 can be any workstation, telephone, desktop computer, laptop or notebook computer, netbook, ULTRABOOK, tablet, server, handheld computer, mobile telephone, smartphone, or other portable telecommunications device, media playing device, a gaming system, mobile computing device, or any other type and/or form of computing, telecommunications or media device that is capable of communication. The computer system 1200 has sufficient processor power and memory capacity to perform the operations described herein. In some embodiments, the computing device 1200 can have different processors, operating systems, and input devices consistent with the device.

In some embodiments, the status of one or more machines 1200 in the network can be monitored, for example, as part of network management. In one of these embodiments, the status of a machine can include an identification of load information (e.g., the number of processes on the machine, CPU, and memory utilization), of port information (e.g., the number of available communication ports and the port addresses), or of session status (e.g., the duration and type of processes, and whether a process is active or idle). In another of these embodiments, this information can be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

Implementations of the subject matter and the operations described in this specification can be implemented in digital electronic circuitry, or in computer software embodied on a tangible medium, firmware, or hardware, including the structures disclosed in this specification and their structural equivalents, or in combinations of one or more of them. Implementations of the subject matter described in this specification can be implemented as one or more computer programs, e.g., one or more components of computer program instructions, encoded on computer storage medium for execution by, or to control the operation of data processing apparatus. The program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can include a source or destination of computer program instructions encoded in an artificially-generated propagated signal. The computer storage medium can also be, or be included in, one or more separate physical components or media (e.g., multiple CDs, disks, or other storage devices 1028).

The operations described in this specification can be implemented as operations performed by a data processing apparatus on data stored on one or more computer-readable storage devices or received from other sources.

The terms “data processing apparatus”, “data processing system”, “client device”, “computing platform”, “computing device”, or “device” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing, and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array), or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The elements of a computer include a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), for example. Devices suitable for storing computer program instructions and data include all forms of non-volatile memory, media, and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube), plasma, or LCD (liquid crystal display) monitor, for displaying information to the user, a keyboard, and a pointing device, e.g., a mouse or a trackball, by which the user can provide input to the computer. Other kinds of devices can be used to provide for interaction with a user as well; for example, feedback provided to the user can include any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface (e.g., the user interface 120 described in connection with FIGS. 1 and 2 or the user interface 1020 described in connection with FIG. 10) or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While this specification contains many specific implementation details, these should not be construed as limitations on the scope of any inventions or of what can be claimed, but rather as descriptions of features specific to particular implementations of the systems and methods described herein. Certain features that are described in this specification in the context of separate implementations can also be implemented in combination in a single implementation. Conversely, various features that are described in the context of a single implementation can also be implemented in multiple implementations separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

In certain circumstances, multitasking and parallel processing can be advantageous. Moreover, the separation of various system components in the implementations described above should not be understood as requiring such separation in all implementations, and it should be understood that the described program components and systems can generally be integrated together in a single software product or packaged into multiple software products.

Having now described some illustrative implementations and implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements, and features discussed only in connection with one implementation are not intended to be excluded from a similar role in other implementations or implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation, and references to “an implementation,” “some implementations,” “an alternate implementation,” “various implementation,” “one implementation,” or the like are not necessarily mutually exclusive and are intended to indicate that a particular feature, structure, or characteristic described in connection with the implementation can be included in at least one implementation. Such terms as used herein are not necessarily all referring to the same implementation. Any implementation can be combined with any other implementation, inclusively or exclusively, in any manner consistent with the aspects and implementations disclosed herein.

References to “or” can be construed as inclusive so that any terms described using “or” can indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein can be embodied in other specific forms without departing from the characteristics thereof. Although the examples provided can be useful navigating surgical robots in accordance with patient movement and surgical environment conditions, the systems and methods described herein can be applied to other environments. The foregoing implementations are illustrative rather than limiting of the described systems and methods. The scope of the systems and methods described herein can thus be indicated by the appended claims, rather than the foregoing description, and changes that come within the meaning and range of equivalency of the claims are embraced therein.

What is claimed is:

1. A method, comprising:
accessing, by one or more processors coupled to memory, a three-dimensional (3D) point cloud corresponding to a surgical environment and a patient, the 3D point cloud having a frame of reference;
determining, by the one or more processors, a position of a surgical robot within the frame of reference of the 3D point cloud;
detecting, by the one or more processors, a change in a position of the patient based on a corresponding change in position of one or more points in the 3D point cloud; and
generating, by the one or more processors, responsive to detecting the change in the position of the patient, instructions to modify the position of the surgical robot based on the change in position of the one or more points.

2. The method of claim 1, wherein determining the position of the surgical robot within the frame of reference further comprises calibrating, by the one or more processors, the surgical robot using a calibration technique.

3. The method of claim 1, wherein the surgical robot further comprises a display positioned over a surgical site in the surgical environment, and wherein the method further comprises presenting, by the one or more processors, an image captured by a capture device mounted on the surgical robot.

4. The method of claim 1, wherein the surgical robot comprises an attachment that receives a surgical tool, and wherein determining the position of the surgical robot further comprises determining, by the one or more processors, a position of the surgical tool.

5. The method of claim 1, further comprising navigating, by the one or more processors, the surgical robot along a predetermined pathway in the frame of reference.

6. The method of claim 5, wherein navigating the surgical robot further comprises:
adjusting, by the one or more processors, a position of the surgical robot according to a predetermined trajectory in the frame of reference,
periodically determining, by the one or more processors, whether the change in the position of the patient satisfies a threshold; and
adjusting, by the one or more processors, the position of the surgical robot according to the predetermined trajectory and the change in the position of the patient responsive to determining that the change in the position of the patient satisfies the threshold.

7. The method of claim 1, wherein determining the position of the surgical robot is based on an infrared tracking technique.

8. The method of claim 7, wherein the surgical robot comprises one or more markers, and wherein determining the position of the surgical robot based on the infrared tracking technique comprises detecting a respective position of each of the one or more markers.

9. The method of claim 1, wherein detecting the change in the position of the patient comprises comparing, by the one or more processors, a point of the 3D point cloud with a second point of a second 3D point cloud captured after the 3D point cloud.

10. The method of claim 9, wherein detecting the change in the position of the patient comprises determining that a distance between the point and the second point exceeds a predetermined threshold.

11. A system, comprising:
one or more processors coupled to a non-transitory memory, the one or more processors configured to:
access a three-dimensional (3D) point cloud corresponding to a surgical environment and a patient, the 3D point cloud having a frame of reference;
determine a position of a surgical robot within the frame of reference of the 3D point cloud;
detect a change in a position of the patient based on a corresponding change in position of one or more points in the 3D point cloud; and
generate, responsive to detecting the change in the position of the patient, instructions to modify the position of the surgical robot based on the change in position of the one or more points.

12. The system of claim 1, wherein the one or more processors are further configured to determine the position of the surgical robot within the frame of reference by performing operations comprising calibrating the surgical robot using a calibration technique.

13. The system of claim 11, wherein the surgical robot further comprises a display positioned over a surgical site in the surgical environment, and wherein the one or more processors are further configured to present an image captured by a capture device mounted on the surgical robot.

14. The system of claim 11, wherein the surgical robot comprises an attachment that receives a surgical tool, and wherein the one or more processors are further configured to determine a position of the surgical tool.

15. The system of claim 11, wherein the one or more processors are further configured to navigate the surgical robot along a predetermined pathway in the frame of reference.

16. The system of claim 15, wherein to navigate the surgical robot, the one or more processors are further configured to:
adjust a position of the surgical robot according to a predetermined trajectory in the frame of reference;
periodically determine whether the change in the position of the patient satisfies a threshold; and
adjust the position of the surgical robot according to the predetermined trajectory and the change in the position of the patient responsive to determining that the change in the position of the patient satisfies the threshold.

17. The system of claim 11, wherein the one or more processors are further configured to determine the position of the surgical robot based on an infrared tracking technique.

18. The system of claim 17, wherein the surgical robot comprises one or more markers, and wherein the one or more processors are further configured to detect a respective position of each of the one or more markers.

19. The system of claim 11, wherein the one or more processors are further configured to detect the change in the position of the patient by performing operations comprising comparing a point of the 3D point cloud with a second point of a second 3D point cloud captured after the 3D point cloud.

20. The system of claim 19, wherein the one or more processors are further configured to detect the change in the position of the patient by performing operations comprising determining that a distance between the point and the second point exceeds a predetermined threshold.
